(19) United States
(12) Patent Application Publication
(10) Pub. No.: US 2022/0387187 A1
(43) Pub. Date: Dec. 8, 2022

(54) Implant Selection And Surgical Planning Based On Patient Specific Kinematics

(71) Applicant: Howmedica Osteonics Corp., Mahwah, NJ (US)

(72) Inventors: Xiangyi Liu, Mahwah, NJ (US); Azhar Ali, West Orange, NJ (US); Jerry D'Alessio, Cedar Grove, NJ (US); Ronald Ehlert, Houston, TX (US)

(73) Assignees: Howmedica Osteonics Corp., Mahwah, NJ (US); Howmedica Osteonics Corp., Mahwah, NJ (US)

(21) Appl. No.: 17/834,163

(22) Filed: Jun. 7, 2022

Related U.S. Application Data
(60) Provisional application No. 63/208,211, filed on Jun. 8, 2021.

(51) Int. Cl. A61F 2/46; G16H 20/40

(52) U.S. Cl. CPC A61F 2/461; G16H 20/40; A61F 2002/4633

(57) ABSTRACT

In one embodiment, a method of planning implantation of a knee prosthesis is performed. Data is collected that is representative of a position of a tibia relative to a femur at a plurality of positions of the tibia relative to the femur through a range of motion of the knee. The data at each position of the plurality of positions includes: a medial maximum convergence of a low point on a medial femoral condyle and a medial tibial articular surface of the tibia and a lateral maximum convergence of a low point on a lateral femoral condyle and a lateral tibial articular surface of the tibia. The medial and lateral convergence locations from the plurality of positions are analyzed to determine a kinematic pattern that is then used to determine an implant position and an implant orientation for a tibial implant to be placed on a resected tibial surface.

### CROSS-REFERENCE TO RELATED APPLICATION

This application claims the benefit of the filing date of U.S. Provisional Patent Application No. 63/208,211, filed on Jun. 8, 2021, the disclosure of which is hereby incorporated by reference herein in its entirety.

### BACKGROUND OF THE INVENTION

Various procedures are known for total knee arthroplasty or for placement of implants in parts of the knee joint to remedy damage to the knee or disease in the knee. In some instances, the implants used in these procedures may be designed to account for kinematics specifically associated with the knee joint in its flexion range of motion. For example, most individuals exhibit a medial pivot in deep flexion where a lateral side of the knee joint rotates about the medial side through a range of motion. Designs of tibial implants may or may not account for this dynamic relationship.

Further, although some tibial implants have been designed with consideration given to knee kinematics, such designs do not account for all the various types of kinematic patterns in the knee joint. For instance, a tibial implant designed with a view to a medial pivot pattern will not accommodate a lateral pivot pattern. In this manner, an individual that does not naturally possess medial pivot kinematics would be forced into the medial pivot kinematic pattern and could, through a range of motion experienced during everyday activity, encounter conflicts with soft tissue or other restraints through at least part of the range of motion. Ultimately, this may cause stiffness, loss of range of motion, pain, and/or instability.

In addition to the foregoing, existing technologies fail to contemplate a tibial implant plan to address requirements based on an individual's actual kinematics in the knee. As a result, even when an individual with medial pivot kinematics receives an implant that is designed with a view to a medial pivot pattern in the joint, such implant may not have a pivot center that matches the individual's actual pivot center. As above, such deficiencies in existing designs may, when implemented, cause stiffness, loss of range of motion, pain, and/or instability.

Thus, a need exists for improvements to planning knee replacement surgery to account for a range of natural and individual kinematic patterns in the knee joint and, for a particular type of kinematic pattern, to account for the particular natural kinematic pattern of the individual.

### BRIEF SUMMARY OF THE INVENTION

In one aspect, the present disclosure relates to a method of planning implantation of a tibial implant in a patient. In one embodiment, the planning method includes: in response to rotation of a tibia of the patient through a range of motion relative to a femur of the patient, the range of motion including at least part of a distance between extension of the knee and flexion of the knee, collecting data representative of a position of the tibia relative to the femur at a plurality of orientations of the tibia relative to the femur such that data is collected for a plurality of positions, the data at each position of the plurality of positions including: a medial contact location defined by a maximum convergence of a low point on a medial condyle of the femur and a medial tibial articular surface of the tibia; and a lateral contact location defined by a maximum convergence of a low point on a lateral condyle of the femur and a lateral tibial articular surface of the tibia; analyzing the medial and lateral contact locations from the plurality of positions to determine a kinematic pattern; and utilizing the kinematic pattern to determine an implant position and an implant orientation for a tibial implant to be placed on a resected tibial surface.

In some embodiments, the kinematic pattern may be in a transverse plane and include a plurality of lateral contact locations and a plurality of medial contact locations, both inclusive of the plurality of positions. The plurality of lateral contact locations may define a lengthwise sequence along the lateral tibial articular surface, and the plurality of medial contact locations may collectively define a pivot point on the medial tibial articular surface such that the tibia rotates about the pivot point as the knee joint moves between extension and flexion. In some embodiments, determining the implant position of the tibial implant may involve centering a medial sulcus point of a medial articular surface of the tibial implant on the pivot point. In some embodiments, determining the orientation of the tibial implant may involve rotating the tibial implant about the pivot point such that the plurality of lateral contact locations are all interior to a perimeter of a lateral articular surface of the tibial implant.

In some embodiments, the method may include selecting at least one of a size of the tibial implant and surface characteristics of the tibial implant based on the kinematic pattern. In some embodiments, the method may include modifying a planned resection cut of the tibia such that the planned resection cut is not entirely flush with a transverse plane, the modification improving correspondence between the kinematic pattern and the tibial implant when the kinematic pattern is overlaid on the tibial implant. In some embodiments, the method may include performing gap balancing of the knee joint at different orientations of the tibia relative to the femur to determine a first cut location on the tibia, the first cut location defining a proximal tibial surface for receipt of the tibial implant.

In some embodiments, the distance between extension of the knee and flexion of the knee may be between hyperextension of the knee and 120 degrees flexion of the knee. In some embodiments, the kinematic pattern may be in a transverse plane and may include a first range of medial contact locations and a second range of lateral contact locations. The first range and the second range may be separated by a pivot point on a first location of an intercondylar eminence of the tibia such that the tibia rotates about the first location as the knee joint moves between extension and flexion, the first location being identifiable relative to medial and lateral sides of the intercondylar eminence. In some embodiments, determining the implant position of the tibial implant may involve centering the tibial implant on the first location of the tibia. In some embodiments, determining the orientation of the tibial implant may involve rotating the tibial implant such that a perimeter of a medial articular surface of the tibial implant envelopes all medial contact locations of the kinematic pattern and a perimeter of a lateral articular surface of the tibial implant envelopes all lateral contact locations of the kinematic pattern.

In some embodiments, the kinematic pattern may be in a transverse plane and may include a plurality of medial contact locations and a plurality of lateral contact locations, both from the plurality of positions. The plurality of medial contact locations may define a lengthwise sequence along the medial tibial articular surface, and the plurality of lateral contact locations may collectively define a pivot point on the lateral articular surface such that the tibia rotates about the pivot point as the knee joint moves between extension and flexion. In some embodiments, the position of the tibial implant may involve centering a lateral sulcus point on a lateral articular surface of the tibial implant on the pivot point. In some embodiments, determining the orientation of the tibial implant may involve rotating the tibial implant about the pivot point such that a periphery of the medial articular surface of the tibial implant envelopes all of the medial contact locations of the kinematic pattern.

In some embodiments, the kinematic pattern may include a plurality of medial contact locations from the plurality of positions and a plurality of lateral contact locations from the plurality of positions. Each of the plurality of medial contact locations and the plurality of lateral contact locations may collectively define a line in an approximately anterior-posterior direction in a transverse plane. In some embodiments, determining the position and orientation of the tibial implant may involve translating and rotating the tibial implant such that a periphery of a medial articular surface of the tibial implant envelopes the plurality of medial contact locations of the kinematic pattern and a periphery of a lateral articular surface of the tibial implant envelopes the plurality of lateral contact locations of the kinematic pattern. In some embodiments, the collecting of data may be performed with a tracking device to monitor the position of the tibia and the femur throughout the range of motion. In some embodiments, the collecting of data may be performed pre-operatively using medical imaging.

In some embodiments, a method of implanting a knee prosthesis may include a method of planning implantation of the knee prosthesis as described for various embodiments herein, followed by: resecting the tibia to prepare the resected tibial surface; obtaining the tibial implant; and positioning the tibial implant on the resected tibial surface according to the determined position and orientation of the tibial implant. In some embodiments, the method of implanting may include collecting data based on movement of the tibia through a second range of motion with the tibial implant positioned and oriented on the resected tibial surface to determine a second kinematic pattern. In some embodiments, obtaining the tibial implant may involve obtaining a trial tibial implant. In some embodiments, utilizing the kinematic pattern to determine the implant position and the implant orientation may be accomplished with the use of a virtual implant overlaid on both a virtual representation of the kinematic pattern and a virtual model of the tibia. In some embodiments, utilizing the kinematic pattern to determine the implant position and the implant orientation may involve overlaying the virtual implant on the virtual model of the tibia inclusive of virtual resections of the tibia, the virtual resections determined based on at least one of the kinematic pattern and gap balancing of the knee joint.

In one aspect, the present disclosure relates to a method of implanting a total knee prosthesis in a patient. In one embodiment, a method of implanting a total knee prosthesis in a patient involves: retrieving data at a plurality of positions of a tibia of the patient relative to a femur of the patient, the plurality of positions collectively representative of at least part of a range of motion of a knee of the patient, wherein the data at each of the plurality of positions includes: a medial contact location defined by a maximum convergence of a low point on a medial condyle of the femur and a first medial tibial articular surface of the tibia; and a lateral contact location defined by a maximum convergence of a low point on a lateral condyle of the femur and a first lateral tibial articular surface of the tibia; analyzing the data collected from the plurality of positions to determine a range of medial contact locations based on the at least part of the range of motion and a range of lateral contact locations based on the at least part of the range of motion; and virtually selecting a virtual tibial implant with a second medial tibial articular surface and a second lateral tibial articular surface; determining a planned implant position for the virtual tibial implant and a planned implant orientation for the virtual tibial implant by positioning and orienting the virtual tibial implant such that the range of medial contact locations are overlaid within the second medial tibial articular surface and the range of lateral contact locations are overlaid within the second lateral tibial articular surface; resecting the tibia to define a resected tibial surface; and placing a tibial implant corresponding to the virtual tibial implant on the resected tibial surface according to the planned implant position and the planned implant orientation.

In some embodiments, the range of lateral contact locations may define a lengthwise sequence in a transverse plane and the range of medial contact locations may collectively define a pivot point in the transverse plane such that the tibia rotates about the pivot point as the knee joint moves between extension and flexion. In some embodiments, determining the planned implant position of the virtual tibial implant may involve centering a medial sulcus point of the medial articular surface of the virtual tibial implant on the pivot point. In some embodiments, determining the planned implant orientation of the virtual tibial implant may involve rotating the virtual tibial implant about the pivot point such that the range of lateral contact locations are overlaid within the second lateral tibial articular surface. In some embodiments, the method may include utilizing the range of medial contact locations and the range of lateral contact locations to determine a resection cut of the tibia such that upon resection, the tibial implant is receivable on the resected proximal tibia surface. In some embodiments, the method may include performing gap balancing of the knee joint at different positions of the tibia relative to the femur to determine a resection cut of the tibia such that upon resection, the tibial implant is receivable on a resected proximal tibia surface.

In some embodiments, the method of implanting the total knee prosthesis in the patient may also include: retrieving implant data at a second plurality of positions of the tibia relative to the femur with the tibial implant positioned on the resected tibial surface, the second plurality of positions collectively representative of at least part of the range of motion. The data at each of the plurality of positions may include the medial contact location and the lateral contact location with the tibial implant in place of the tibia. The method may also include analyzing the implant data collected from the second plurality of positions to determine a second kinematic pattern based on a maximum convergence of the tibial implant and the femur at the second plurality of positions and comparing the second kinematic pattern based on the implant data with a first kinematic pattern including the range of medial contact locations and the range of lateral contact locations.

In some embodiments, the method may include changing at least one of the planned implant position and the planned implant orientation when a difference between the second kinematic pattern and the first kinematic pattern is above a predetermined threshold. In some embodiments, selecting the tibial implant may involve selecting a trial implant.

In one aspect, the present disclosure relates to a method of evaluating motion in a knee joint of a patient where a tibia of the patient includes a tibial implant received thereon. In one embodiment, a method of evaluating motion in a knee joint of a patient where a tibia of the patient includes a tibial implant received thereon includes: collecting data at a plurality of positions of the tibia relative to a femur of the patient, the plurality of positions collectively representative of at least part of a range of motion of the knee and including at least part of a distance between extension of the knee joint and flexion of the knee joint, the data at each position including: a medial contact location defined by a maximum convergence of a low point on a medial condyle of the femur and a first surface location on a medial side of a tibial plateau of the tibial implant; and a lateral contact location defined by a maximum convergence of a low point on a lateral condyle of the femur and a second surface location on a lateral side of the tibial plateau of the tibial implant; analyzing the medial and lateral contact locations from the plurality of positions to determine a kinematic pattern of the knee joint; and determining whether, at one or more positions, the first surface location is outside of a medial tibial articular surface of the tibial implant and whether, at one or more positions, the second surface location is outside of a lateral tibial articular surface of the tibial implant.

In some embodiments, when the first surface location is outside of the medial tibial articular surface at a first position of the plurality of positions, the method may involve identifying the first position as outside of an acceptable range for minimally sufficient function of the knee joint. In some embodiments, the first position being outside of the acceptable range may indicate impingement. In some embodiments, when the second surface location is outside of the lateral tibial articular surface at a first position of the plurality of positions, the method may involve identifying the first position as outside of an acceptable range for minimally sufficient function of the knee joint. In some embodiments, the first position being outside of the acceptable range may indicate impingement.

In some embodiments, the method may also include: when the first surface location is outside of the medial tibial articular surface in at least one position of the plurality of positions or the second surface location is outside of the lateral tibial articular surface in at least one position of the plurality of positions: removing the tibial implant; selecting a second tibial implant based on the kinematic pattern; and implanting the second tibial implant onto the tibia. In some embodiments, the collecting of data may be performed with a tracking device to monitor the position of the tibial implant and the femur throughout the range of motion.

In one embodiment, a method of evaluating motion in a knee joint of a patient where a tibia of the patient includes a tibial implant received thereon includes: retrieving data at a plurality of positions of the tibia relative to a femur of the patient, the plurality of positions collectively representative of at least part of a range of motion of the knee and including at least part of a distance between extension of the knee joint and flexion of the knee joint, the data at each position including: a medial contact location defined by a maximum convergence of a low point on a medial condyle of the femur and a first surface location on a medial side of a tibial plateau of the tibial implant; and a lateral contact location defined by a maximum convergence of a low point on a lateral condyle of the femur and a second surface location on a lateral side of the tibial plateau of the tibial implant; analyzing the medial and lateral contact locations from the plurality of positions to determine a second kinematic pattern based on the at least part of a range of motion; comparing the second kinematic pattern with a first kinematic pattern, the first kinematic pattern being determined pre-operatively based on movement of the tibia relative to the femur over the range of motion; and identifying, through the comparison, any differences in the second kinematic pattern relative to the first kinematic pattern.

In some embodiments, the first kinematic pattern may include a medial pivot and differences in the second kinematic pattern relative to the first kinematic pattern may include that the second kinematic pattern has a shorter range of lateral contact locations among the plurality of positions than the first kinematic pattern. In some embodiments, the first kinematic pattern may include a medial pivot and in a first position, the second surface location of a first lateral contact location may be outside of a lateral tibial articular surface of the tibial implant. In some embodiments, the method may include: when the differences between the second kinematic pattern and the first kinematic pattern are greater than a predetermined threshold: removing the tibial implant; selecting a second tibial implant based at least in part on the first kinematic pattern; and implanting the second tibial implant onto the tibia. In some embodiments, the differences between the second and first kinematic pattern may be greater than the predetermined threshold when at a first position from among the plurality of positions, at least one medial or lateral contact location is outside of a respective medial tibial articular surface or lateral tibial articular surface. In some embodiments, the differences between the second and first kinematic pattern may be greater than the predetermined threshold when a second distance representing a range of pivot in the second kinematic pattern is less than 80% of a first distance representing a range of pivot in the first kinematic pattern. In some embodiments, determining the first kinematic pattern and the second kinematic pattern may require the use of a tracking device to monitor the position of the tibial implant and the femur throughout the range of motion.

In one aspect, the present disclosure relates to evaluating motion in a knee joint of a patient for planning placement of a tibial implant on a tibia. In one embodiment, a method of evaluating motion in a knee joint of a patient for planning placement of a tibial implant on a tibia includes: receiving a signal from a robotic arm indicating an establishment of communication with the robotic arm, the robotic arm being engaged with at least one muscle responsive to flexion in the knee joint such that rotation of the tibia relative to a femur of the patient using the robotic arm causes the at least one muscle to be stimulated; collecting data at a plurality of positions of the tibia relative to a femur of the patient based on activation of the robotic arm, the plurality of positions collectively representative of at least part of a range of motion of the knee and including at least part of a distance between extension of the knee joint and flexion of the knee joint, the data at each position including: a medial contact location defined by a maximum convergence of a low point on a medial condyle of the femur and a first surface location on a medial side of a tibial plateau of the tibial implant; and a lateral contact location defined by a maximum convergence of a low point on a lateral condyle of the femur and a second surface location on a lateral side of the tibial plateau of the tibial implant; analyzing the medial and lateral contact locations from the plurality of positions to determine a kinematic pattern of the knee joint; and determining whether, at one or more positions, there is impingement between either the tibia and soft or hard tissue or the femur and soft or hard tissue. And, when there is impingement, modifying a planned tibial implant position to at least reduce an extent of impingement.

In one aspect, the present disclosure relates to a method of determining a femoral implant placement on a femur in a knee joint of a patient. In one embodiment, a method of determining a femoral implant placement on a femur in a knee joint of a patient includes: collecting data at a plurality of positions of a tibia of the patient relative to the femur, the plurality of positions collectively representative of at least part of a range of motion of the knee and including at least part of a distance between extension of the knee joint and flexion of the knee joint, the data at each position including: a dynamic flexion axis of the femur, the dynamic flexion axis being a center of rotation of a length of the tibia about the femur; calculating a reference dynamic flexion axis based on the dynamic flexion axis of the femur in at least two positions of the plurality of positions; and planning the femoral implant placement on the femur based on the reference dynamic flexion axis.

In some embodiments, planning may include analyzing the femoral implant placement in each of the coronal plane, the sagittal plane and the transverse plane. In some embodiments, calculating of the reference dynamic flexion axis may be based on an average of the dynamic flexion axis in the at least two positions of the plurality of positions.

In one aspect, the present disclosure relates to a method of planning implantation of a knee prosthesis in a patient. In one embodiment, the method includes the following steps. In response to rotation of a tibia of the patient through a range of motion relative to a femur of the patient, the range of motion including at least part of a distance between extension of the knee and flexion of the knee, data is collected that is representative of a position of the tibia relative to the femur at a plurality of orientations of the tibia relative to the femur. The data is collected for a plurality of positions. The data at each position of the plurality of positions includes a medial contact location and a lateral contact location. The medial contact location is defined by one of a maximum convergence of a low point on a medial side of the femur and a surface of the tibia or a location of maximum convergence between the femur and the tibia on the medial side. The lateral contact location is defined by one of a maximum convergence of a low point on a lateral side of the femur and a surface of the tibia or a location of maximum convergence between the femur and the tibia on the lateral side. The medial and lateral contact locations from the plurality of positions are analyzed to determine a kinematic pattern. And, the kinematic pattern is utilized to determine an implant position and an implant orientation for a tibial implant to be placed on a resected tibial surface.

In some examples, only one of the medial contact location and the lateral contact location may be defined by a maximum convergence of a low point on the femur and a surface of the tibia. In other examples, only one of the medial contact location and the lateral contact location may be defined by a location of maximum convergence between the femur and the tibia on the medial or lateral side, respectively. In still further examples, one of the medial contact location and the lateral contact location may be defined by a maximum convergence of a low point on the femur and a surface of the tibia and the other of the medial contact location and the lateral contact location may be defined by a location of maximum convergence between the femur and the tibia. In certain of the above examples, the low point on the femur may be a femoral condyle. In still further examples, each of the medial contact location and the lateral contact location may be defined in the same way.

In one aspect, the present disclosure relates to methods of improving tibial implant selection and positioning on a resected tibia based on the unique traits of the patient at issue. And, in particular, taking into consideration not only the physical shape of the anatomy in the knee, but also the kinematic characteristics of the knee joint. Further, the present disclosure provides a way to realize these improved methods in an intraoperative setting and may be performed, if desired, with minimal instrumentation and other accessories. For example, in a first embodiment, a method may involve placing a platform with a sheet thereon into a knee joint, and, more specifically, onto a proximal surface of the tibia. The knee may then be moved through a range of motion such that contact points between the femoral condyles and the tibial surface, via the sheet, are physically marked onto the sheet. This may be via ink, for example. The imprint left on the sheet represents the kinematic pattern of the patient. Such pattern may be used to determine the type of implant best suited for the patient and to determine the position and orientation of the implant when the implant is ultimately placed on the tibia after resection.

In one aspect, the present disclosure relates to a method of selecting an implant and determining an implant placement location within a joint. In a second embodiment, a method of determining a position for placement of an implant in a joint includes: beginning with an instrument attached to a first bone and a sheet of the instrument positioned over a joint-facing end surface of the first bone, simultaneously collecting a plurality of lateral contact points and a plurality of medial contact points between the first bone and a second bone facing the first bone while the first bone is moved through a range of motion relative to the second bone, the contact points being collected by the sheet; and determining a lateral center based on a geometric center of the plurality of lateral contact points on the sheet and a medial center based on a geometric center of the plurality of medial contact points on the sheet. In the method, the lateral center is relied on for positioning a lateral sulcus of an implant and the medial center is relied on for positioning a medial sulcus of the implant, the implant being adapted for implantation onto the first bone.

In some examples of the second embodiment, the method may also include recognizing, subsequent to placement of the implant onto the first bone, that the lateral center is aligned with the lateral sulcus and the medial center is aligned with the medial sulcus. In other examples, when an outer periphery of the implant overhangs the first bone and removal of the implant is followed by placement of a second implant onto the first bone, recognizing a position and orientation of a second implant as aligned with the first bone. The alignment is recognized when the lateral center is aligned with a second lateral sulcus of the second implant and the medial center is aligned with a second medial sulcus of the second implant. In further examples, the method may also include determining an implant type based on the plurality of lateral contact points on the sheet and the plurality of medial contact points on the sheet, the implant type being one of a medial pivot, a lateral pivot, a central pivot or no pivot. In some examples, the first bone may be resected prior to attachment of the instrument to the first bone. In still further examples, the method may also include allowing an upper platform of the instrument with the sheet thereon to rise relative to a lower platform of the instrument positioned over a resected surface of the end of the first bone prior to the collecting step. In some examples, collecting the plurality of lateral contact points and the plurality of medial contact points between the first bone and a second bone may be based on detection of contact by a plurality of sensors in the sheet. In a subset of these examples, the method may also include receiving a signal from the sensors at a computer in communication with the sensors, the computer being adapted to generate a visual display of the plurality of lateral contact points and the plurality of medial contact points. In some examples, simultaneously collecting the plurality of lateral contact points and the plurality of medial contact points may occur between a tibia and a femur. And, in some examples, the implant may be a trial.

In a third embodiment, a method involves selecting and positioning a tibial implant. Steps of the method include: recording contact points between a femur of a patient and a tibia of the patient as the tibia is moved through a range of motion relative to the femur, the contact points being recorded by a sheet disposed on the tibia and the contact points including a lateral path of contact points and a medial path of contact points; determining a lateral center of a lateral elongate dimension of the lateral path of contact points and a medial center of a medial elongate dimension of the medial path of contact points; generating a display of the lateral center and the medial center on a virtual model of the tibia; and, in response to bringing a tibial implant into proximity with a proximal end of the tibia, displaying the tibial implant on the virtual model, the lateral center and the medial center being visible on the virtual model when the tibial implant is positioned directly over the lateral center and the medial center.

In some examples of the third embodiment, the sheet recording the contact points may be part of an instrument positioned on the tibia. In some examples, the method may also include receiving the contact points recorded by the sheet on a computer, the computer being in communication with the sheet or a tool adapted to identify the lateral path of contact points and the medial path of contact points on the sheet. In other examples, the method may also include determining the lateral center and the medial center by the computer based on the respective lateral path of contact points and the medial path of contact points. In some examples, generation of the display of the virtual model may be performed by the computer. In other examples, the method may also include tracking real-time locations of the tibia and the tibial implant with a navigation system in communication with the computer, the virtual model representing the real-time locations. In some examples, tracking real-time locations of the tibia and the tibial implant may involve communication among one or more cameras, two or more fiducial markers and the computer.

In further examples of the third embodiment, the method may also include receiving the recorded contact points on the computer via a signal transmitted by sensors in the sheet, the sensors being adapted to detect locations of contact between the femur and the tibia. In still further examples, prior to recording contact points, an upper platform of the instrument with the sheet disposed thereon may move into or remain in a biased position remote from a lower platform of the instrument abutting the tibia. In some examples, the method may also include displaying a final implanted position of the tibial implant when the virtual model displays a lateral sulcus of the tibial implant in alignment with the lateral center and a medial sulcus of the tibial implant in alignment with the medial center in the virtual model. In some examples, the tibial implant may be a trial.

In a fourth embodiment, a method involves selecting and aligning a tibial implant. Steps of the method include: positioning an instrument on a tibia of a patient, the instrument including a body and a sheet attached to the body; bringing an end of the sheet remote from the body over an end surface of the tibia such that the sheet covers at least part of the end surface of the tibia; rotating the tibia relative to a femur of the patient such that contact points between the tibia and the femur are collected by the sheet at a plurality of rotational positions during the rotation, wherein the contact points define a medial path of contact between the tibia and the femur and a lateral path of contact between the tibia and the femur; identifying a medial center of the medial path of contact and a lateral center of the lateral path of contact; and positioning a tibial implant on the tibia by aligning a medial sulcus of the tibial implant with the medial center and by aligning a lateral sulcus of the tibial implant with the lateral center.

In some examples of the fourth embodiment, the method may include selecting the tibial implant based on the medial center, the lateral center and an outer perimeter of the tibia at the end surface. In other examples, the method may include retrieving a second tibial implant different from the tibial implant when an outer profile of the tibial implant aligned with the medial and lateral centers is larger than an outer profile of the end surface of the tibia. In still further examples, rotating the tibia relative to a femur may cause the contact points to be physically marked onto the sheet. In some examples, the method may include positioning the tibial implant on the tibia through a visual inspection of the medial sulcus relative to the medial center on the sheet and the lateral sulcus relative to the lateral center on the sheet based on a translucency or transparency of the tibial implant. In further examples, the method may include detecting contact between the tibia and the femur through sensors in the sheet, the sensors transmitting a signal to a computer to store the medial path of contact and the lateral path of contact. In some examples, the method may include storing the medial center, the lateral center and a virtual model of the tibia on a computer and using a navigation system to monitor a location of the tibial implant in real-time so that the positioning of the tibial implant on the tibia is displayed on a user interface connected to the computer. In some examples, the tibial implant may be a trial.

In another aspect, the present disclosure relates to a positioning instrument for use in collecting patient-specific information about movement in a joint. In a fifth embodiment, the positioning instrument includes a body, a sheet attached to the body, and an anchorage attached to the body. The sheet may be made of a biocompatible material and adapted to record locations of surface contact on the sheet based on an object contacting the sheet. The anchorage may be adapted to engage with a bone.

In some examples of the fifth embodiment, the sheet may be configured to have a first appearance without being contacted by an object and a second appearance when contacted by the object, the second appearance being different from the first appearance. In other examples, the sheet may include a plurality of sensors, each of the plurality of sensors adapted to emit a signal when the object contacting the sheet is proximal to the sensor. In some examples, a platform may be attached to the body, the platform oriented transversely relative to an elongate direction of the body and configured such that the sheet is disposable directly onto the platform when the platform is positioned on a bone. In variations of these examples, the instrument may also include a resilient member. The resilient member has a first end attached to the platform and a second end attached to an upper platform of the instrument, the instrument being arranged such that when the resilient member is compressed, the upper platform is adjacent to the platform and when the resilient member is expanded, the upper platform is remote from the platform.

In a sixth embodiment, a system may include the positioning instrument of the fifth embodiment and navigation tools. In one example, the system includes the positioning instrument, a camera, a fiducial marker, a probe and a computer. The computer may be adapted to communicate with the camera, fiducial marker and probe to monitor a real-time location of a bone and the probe. In some examples, when the bone that receives the anchorage is a tibia and the tibia is moved through a range of motion relative to a femur, the sheet is adapted to record a medial lateral path of contact and a lateral path of contact. In some examples, the computer may be adapted to collect and store data points representative of the lateral path of contact and the medial path of contact.

In a seventh embodiment, a positioning instrument is the same as that described for the fourth embodiment except that the body with anchorage is replaced by a handle. In some examples, such positioning instrument may include pins or other small anchorages on the platform to secure the instrument to the bone.

### BRIEF DESCRIPTION OF THE DRAWINGS

The foregoing summary, as well as the following detailed description of the embodiments of the disclosure, will be better understood when read in conjunction with the appended drawings.

Referring to the figures, wherein like reference numerals represent like elements throughout the several views:

FIG. 1 is a side view of a knee joint being moved through a range of motion according to one embodiment of the disclosure.

FIG. 2 is a top view of a tibial plateau with indications of various possible kinematic patterns in a knee joint based on a range of motion in the knee joint.

FIG. 3 is a diagram illustrating different types of kinematic patterns in a knee joint based on movement of the knee joint through a range of motion.

FIG. 4 is a top view of a tibial plateau with indications of a first medial pivot kinematic pattern established according to a step in a method of one embodiment of the disclosure.

FIG. 5 is a top view of the tibia with a tibial implant virtually overlaid thereon in a first orientation according to another step in the method embodiment of FIG. 4.

FIG. 6 is a top view of the tibia with the tibial implant virtually overlaid thereon in a second orientation according to another step in the method embodiment of FIG. 4.

FIG. 7 is a side view of the knee joint with a tibial implant implanted on the tibia according to one example of the method embodiment of FIG. 4, the knee joint being moved through a range of motion.

FIG. 8 is a top view of the tibial implant with indications of a second medial pivot kinematic pattern according to one example of the method embodiment of FIG. 4.

FIGS. 9A-B are front and side views, respectively, of a dynamic flexion axis at different orientations of the tibia relative to the femur according to one embodiment of the disclosure.

FIGS. 10A-B are front and side views, respectively, of a femoral knee center axis according to the embodiment of FIGS. 9A-B.

FIG. 11 is a perspective view of a positioning instrument according to one embodiment of the disclosure.

FIG. 12 is a perspective view of a positioning instrument according to one embodiment of the disclosure.

FIG. 13 is a perspective view of a positioning instrument according to one embodiment of the disclosure.

FIG. 14 illustrates a step of instrument positioning in a method of selecting and positioning a tibial implant according to one embodiment of the disclosure.

FIG. 15 illustrates a step of rotating the knee joint according to the method of FIG. 14.

FIG. 16 illustrates a step of evaluating data collected from rotating the knee according to the method of FIG. 14.

FIG. 17 illustrates a step of positioning a tibial implant according to the method of FIG. 14.

FIG. 18 illustrates a step of instrument positioning in a method of selecting and positioning a tibial implant according to one embodiment of the disclosure.

FIG. 19 illustrates a step of rotating the knee joint according to the method of FIG. 18.

FIGS. 20 and 21 illustrate different stages of inserting a tibial implant into the knee joint according to the method of FIG. 18.

### DETAILED DESCRIPTION

Reference will now be made in detail to the embodiments of the present disclosure. Wherever possible, the same or like reference numbers will be used throughout the drawings to refer to the same or like features. It should be noted that the drawings are in simplified form and are not drawn to precise scale.

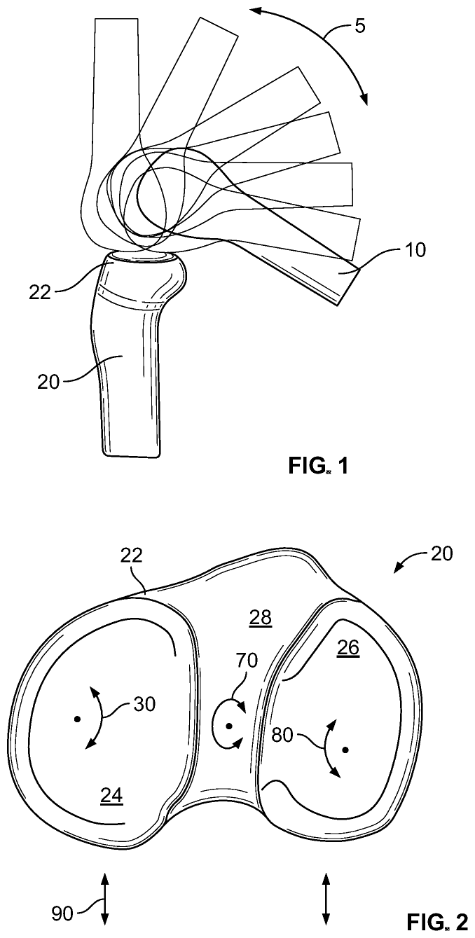

In one aspect of the disclosure, a method of planning an implantation of a tibial implant is contemplated. Generally, the planning method commences with an evaluation of a patient's knee. In particular, a knee of a patient is placed through a range of motion, such as range of motion 5 shown in FIG. 1, in order to obtain paths of maximum convergence locations between a femur 10 and a tibia 20 through range of motion 5. These convergence locations each represent where a femoral low point is closest to a tibial plateau. Collectively, a series of the maximum convergence locations, located on both medial and lateral sides of the joint, is also referred to as a kinematic pattern.

In some embodiments, the kinematic pattern may be obtained pre-operatively while in other embodiments the kinematic pattern may be obtained intra-operatively. In a pre-operative context, techniques including fluoroscopy, x-ray imaging such as early onset scoliosis imaging, or other medical imaging techniques may be used to obtain the kinematic pattern. In particular, images are taken at different degrees of flexion in the knee until data is sufficient to establish the kinematic pattern. When robotics or navigation tools are used, the kinematic pattern may be obtained intra-operatively. In both pre-operative and intra-operative contexts, a tracker may be placed on one or both of the femur and tibia such that locations on surfaces of the femoral condyles and the tibial plateau may be registered. In this manner, such locations may be monitored via the tracker or trackers. Through this arrangement, the knee joint may be moved through a range of motion so that a computer connected to the trackers may collect data at different positions, where collectively the data is representative of the kinematic pattern. Trackers may be in the form of fiducial markers such that bone surface locations desired to be monitored may be registered in conjunction with the use of the fiducial markers. Yet another possibility for obtaining a kinematic pattern involves visual imaging based motion capture, which may be employed either pre-operatively or intra-operatively without the use of x-ray imaging or trackers. In some examples, visual imaging based motion capture may use cameras and/or ultrasound to capture the dynamic characteristics of the joint. In some examples, cameras used may incorporate technology that senses spatial position. In some examples, visual imaging based motion capture may involve the use of machine learning to process data of a patient. In this manner, a neural network may be developed through the collection of a large volume of data, either through a single patient or through many different patients, which in turn may be utilized when processing data obtained for a particular patient. In one implementation, the data in the neural network may contribute to an output of an ultrasound.

In an intra-operative setting, the range of motion may be performed in various ways. For example, a surgeon may manually move the knee through a range of motion. In other examples, a robot arm may be used to move the knee through a range of motion. In a variation on examples that utilize a robot arm, the robot arm may be structured and programmed so that muscles that would be activated when a patient actively moves his or her knee through a range of motion, such as a quadriceps muscle, would be activated. In this manner, the robot may move the knee through a range of motion in a manner that more closely reflects an active range of motion rather than a passive range of motion. Additionally, use of a robot arm may allow for tailoring the number of degrees of freedom in the knee joint that are left open. Such versatility with constraints in the joint may allow for soft tissue guided motion when moving the joint through a range of motion.

The kinematic pattern representative of the range of motion in the knee joint and procured through approaches such as those described above may vary from one patient to another. These kinematic patterns generally represent a natural behavior of a patient's knee through a range of motion, although it should be appreciated that disease or injury may influence such natural behavior. In some embodiments, a knee of a patient is in a condition such that existing natural behavior is inadequate to identify a kinematic pattern. In such cases, the method may include a step of retrieving a kinematic pattern based on previously procured data not associated with the patient. Such kinematic patterns may be based on, for example, a statistical analysis or a type of disease or injury. In some examples, a selection process may be surgeon-directed based solely on judgment or it may rely on other anatomical characteristics of the patient. In other examples, an algorithm may be established based on statistical analysis or other background data that may be processed based on one or more characteristics of a particular patient's existing condition and so that a kinematic pattern may be provided based on an output of the algorithm.

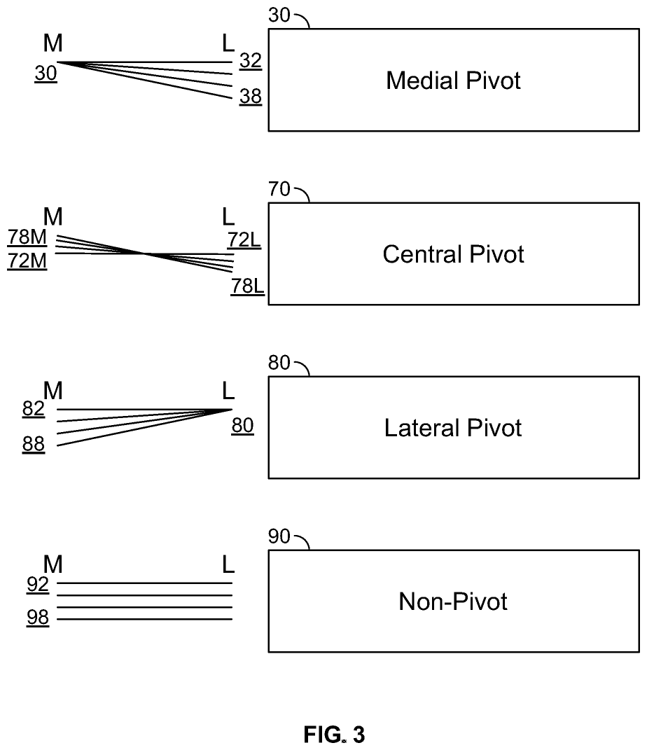

As shown in FIG. 2, in some examples, a patient may have a medial pivot 30, where a location of maximum convergence of a medial femoral condyle (not shown) and a medial tibial articular surface 24 on a tibial plateau 22 of the tibia remains generally the same as the knee moves between extension and flexion, while a location of a maximum convergence of a lateral femoral condyle (not shown) and a lateral articular surface 26 on the tibial plateau 22 changes during the movement. One example of this pattern is shown in FIG. 3, where a location of medial convergence between the femur and tibia remains generally the same while a location of lateral convergence between the femur and tibia moves in a posterior direction as the flexion in the knee joint moves from extension 32 to flexion 38.

In other examples, a patient may have the opposite of the medial pivot: A lateral pivot 80, where a location of a maximum convergence of the lateral femoral condyle and the lateral tibial articular surface remains generally the same as the knee moves between extension and flexion, while a location of a maximum convergence of the medial condyle and the medial articular surface changes during the movement. One example of this pattern is shown in FIG. 3, where a location of lateral convergence between the femur and tibia remains generally the same while a location of medial convergence between the femur and tibia moves in a posterior direction as the flexion in the knee joint moves from extension 82 to flexion 88.

In still other examples, a patient may have a central pivot 70, where a maximum convergence of the femur and tibia on both the medial and lateral sides of the joint changes as the knee moves through a range of motion. The pattern of movement with the central pivot is such that movement on the medial side is in an opposite direction to movement on the lateral side. In this manner, the central pivot 70 is found in a region of an intercondylar eminence of the tibia. One example of this is shown in FIG. 3. When the knee is in extension, a location 72M of maximum convergence on the medial side is more posterior while a location 72L of maximum convergence on the lateral side is more anterior. When the knee joint is rotated into flexion, a location 78M of maximum convergence on the medial side is more anterior, while a location 78L of maximum convergence on the lateral side is more posterior. In other circumstances, a patient may have an opposite kinematic pattern where in extension, maximum convergence on the medial side is anterior while maximum convergence on the lateral side is posterior. Thus, as the above examples illustrate, it should be appreciated that the pivot location established for a medial pivot 30, lateral pivot 80 or central pivot 70 is a center of rotation of the tibial plateau relative to the femur as the knee moves through a range of flexion angles.

In still further examples, a patient may not have a noticeable pivot in the relationship between the femur and the tibia as the knee joint is moved through a range of motion. In such cases, locations of maximum convergence between the bones will generally translate in an anterior-posterior direction as indicated by reference numeral 90 in FIG. 2. More particularly, the locations will translate in a posterior direction as the knee moves from extension 92 to flexion 98, as shown in one example in FIG. 3.

The present disclosure contemplates that data may be collected about a joint of a patient through movement of the joint through a range of motion to determine details of the patient's kinematic pattern. Details of the kinematic pattern may be collected regardless of whether it is a medial pivot 30, central pivot 70, lateral pivot 80 or no pivot 90. This collected data may then be used in accordance with the embodiments of the disclosure to optimize virtual planning of a tibial implant design and placement in the joint, and physical placement in a knee in accordance with the plan. Optimization may involve one or more of positioning the implant, orienting the implant and determining a type and/or size of the implant. In some examples, an implant type may be one of posterior-stabilized, cruciate-retaining, unicompartmental or medial-stabilized. Further, the kinematic pattern may also inform how a proximal tibia should be resected to best accommodate the patient's individual natural kinematics.

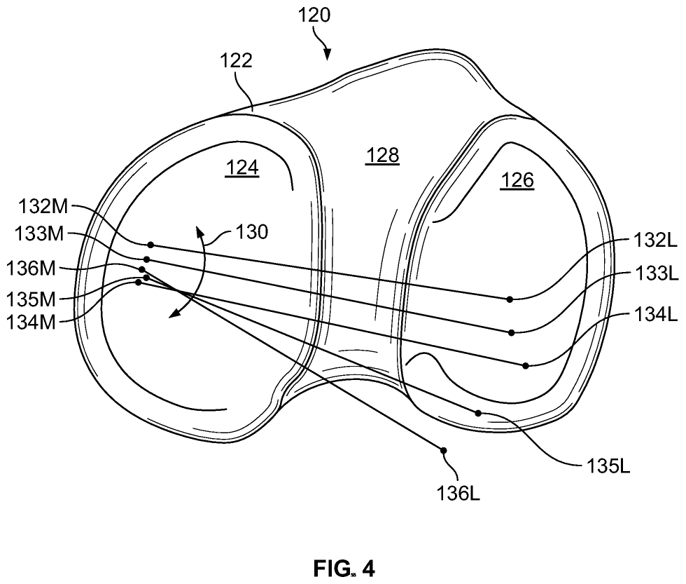

In one embodiment, a method of planning an implantation of a knee prosthesis is as follows. Initially, a kinematic pattern of the knee joint must be determined. This is accomplished through a screening process where a knee of a patient is placed through a range of motion as shown in FIG. 1, either pre-operatively or intra-operatively, with data regarding a position of the tibia relative to the femur collected through medical imaging or tracking tools as described above. In an illustrative example of this embodiment, the movement of the joint through the range of motion establishes that the patient has a kinematic pattern in the form of a medial pivot 130 as shown in FIG. 4. And, the remaining steps in the described method proceed based on the patient having the medial pivot shown. However, it should be appreciated that this is merely one possibility of a patient's anatomical characteristics from among the many described herein. And, the present method may be employed advantageously regardless of the type of kinematic pattern in issue.

The data collected regarding the kinematic pattern of the knee of the patient is stored along with data inclusive of details of the patient's anatomy, including the tibia and the femur. In a variation, the details of the patient's tibia and femur may be originally acquired prior to the range of motion procedure through the use of medical imaging. In some examples, the data is stored electronically on a computer storage device and may be accessed in both visual and numerical form using software. In this manner, a user may retrieve and view the kinematic pattern of the patient overlaid on the knee joint, and in particular, over the tibial plateau, to assess an existing condition of the knee and to develop a plan for tibial implant placement and securement on a proximal end of the tibia. In one example, one view made available to the user shows the kinematic pattern and tibial plateau in the transverse plane. In some examples, the data that collectively represents the kinematic pattern may be processed by software to identify what type of kinematic pattern it is, such as medial pivot, for instance. The software may employ an algorithm to process the data to identify the type. In a specific example, if an anterior-posterior translation of lateral convergence points exceeds 1.5 times the anterior-posterior translation of medial convergence points, the pivot pattern may be determined to be a medial pivot.

Returning to the performance of the described embodiment of the method, after moving the knee joint through the range of motion, the user retrieves and then views the kinematic pattern, here, medial pivot 130, overlaid on the tibial plateau 22 in a transverse plane, as shown in FIG. 4. When software is used for retrieval, the user views the kinematic pattern and tibial anatomy on a user interface, such as a monitor.

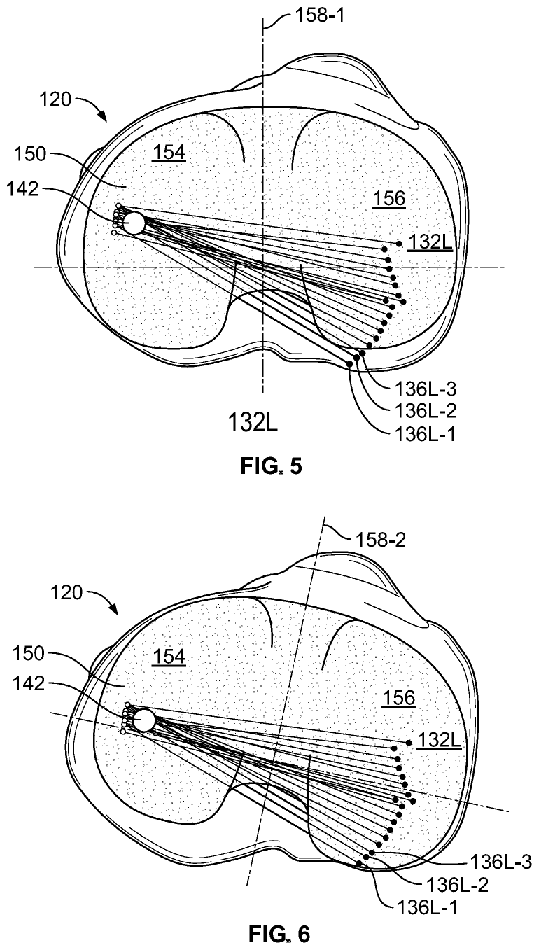

Analysis of the existing conditions is facilitated through the visual display such as that shown in FIG. 4. The user reviews the convergence locations, which may appear as points, on both the medial and lateral sides of the knee, and in orientations of the joint from extension to flexion. Here, these convergence points include medial maximum convergence points 132M-136M and lateral maximum convergence points 132L-136L. It should be appreciated that although FIG. 4 only shows five sets of convergence points, this is for purposes of making the kinematic pattern clear and for ease of explanation. A larger quantity of convergence points defining the kinematic pattern of this embodiment is shown in FIGS. 5-6, inclusive of the points shown in FIG. 4. It is further contemplated that the method may be performed with any number of sets of convergence points, though it is understood that the accuracy of the determined patient kinematics may improve concomitantly with an increase in the number of procured sets of convergence points or with an increase in the range of motion encapsulated by the sets of convergence points.

To plan for positioning a tibial implant on the tibia, the medial pivot point for medial pivot 130 is determined. The kinematic pattern of FIG. 4 is a medial pivot since convergence points on a medial side of the tibial plateau remain relatively static throughout the range of motion. To determine a medial pivot point 142, an average position is calculated based on a totality of medial maximum convergence points 132M, 133M, 134M, 135M, 136M, and other medial convergence points as identified through the range of motion. Of course, certain points may be excluded from the calculation if well outside the range of the others. Medial pivot point 142 is located on a medial articular surface 124 of the tibia, as shown in FIG. 5.

While continuing to view the kinematic pattern on the tibial plateau, a virtual, i.e., computer-generated tibial implant 150 is selected for virtual placement on the tibia 20. The implant may be automatically selected by software based on the tibial anatomy, kinematic pattern, or both, or it may be manually selected by the user. In some examples, an existing condition of the patient may also be used to determine an implant type, such as those described elsewhere in the present disclosure. Upon selection, a sulcus of the medial articular surface 154 of tibial implant 150 is centered on medial pivot point 142, as shown in FIG. 5. This pins tibial implant 150 to the tibia at medial pivot point 142 to establish a planned position of tibial implant 150 on the tibia.

Once the tibial implant 150 is pinned to tibia 20, an orientation of tibial implant 150 is evaluated and considered. Possible orientations are based on rotation of tibial implant 150 about the sulcus of the medial articular surface, which is pinned to medial pivot point 142, as described above. To optimize the orientation of tibial implant 150, lateral maximum convergence points 132L-136L are compared to a preliminary tibial implant 150 location, such as the implant location shown in FIG. 5 where the implant is aligned with a baseline orientation 158-1.

In FIG. 5, several lateral convergence points 136L-1, 136L-2, 136L-3 are shown as outside of the preliminary implant orientation, e.g., baseline orientation 158-1. To bring a full range of the lateral convergence points within a lateral articular surface 156 of the tibial implant 150, the tibial implant is rotated from baseline orientation 158-1 shown in FIG. 5 to a planned orientation 158-2 shown in FIG. 6. As already described, the tibial implant remains pinned to medial pivot point 142 as it is rotated about point 142 from orientation 158-1 to orientation 158-2. In the planned orientation 158-2, lateral convergence points 136L-1, 136L-2, 136L-3 are all directly on lateral articular surface 156. It should be appreciated that some latitude may exist on an exact angle of planned orientation 158-2 based on multiple possibilities that capture a full range of lateral convergence points on lateral articular surface 156. Once the tibial implant is pinned and oriented, the planned implant position and orientation may be evaluated to assess the sufficiency of the plan in covering the full natural range of motion of the patient so that all convergence points are within respective medial and lateral articular surfaces of implant 150. And, based on such evaluation, further adjustments to the tibial implant orientation may be made as appropriate. This completes planning of a tibial implant position and orientation on a tibia.

In variations of the above described embodiment of a planning method, the planning method may be performed on a patient with another type of kinematic pattern based on a flexion range of motion in the knee joint. Thus, although the embodiment shown in FIGS. 4-8 and described in detail above is directed to planning and design for a medial pivot pattern, it should be appreciated that the principles set forth through that embodiment are equally applicable to other patient kinematic patterns. For example, the present disclosure also contemplates determination of a tibial implant position and orientation for circumstances where a patient has natural knee kinematics characterized by a central pivot, lateral pivot or no observable pivot. Examples of these kinematic patterns are shown in FIG. 3. The principles described above for the medial pivot would be applied in the same manner for these alternative natural conditions to arrive at an optimal tibial implant position and orientation based on the applicable kinematic pattern.

The method of planning the implantation of a knee prosthesis may be varied in many ways. In some embodiments, after moving a knee joint of a patient through a range of motion, the kinematic pattern overlaid on the tibial plateau may be viewed to evaluate whether any restrictions on motion exist, whether there is impingement or whether there are other aberrations or deviations from an expected pattern at any position within an expected range of motion. These problematic conditions may also be identified by specific location on the anatomy. For instance, there may be impingement in the right knee on the lateral condyle at 70 degrees flexion, where a maximum convergence point between the lateral condyle and tibial surface is at a particular set of coordinates. More broadly, the knee may be visualized so that colors or other highlighting may be used to show areas of impingement at different degrees of flexion in the knee. The areas of impingement may be shown where they would occur on the tibia in the transverse plane, for example. And, recognition and visualization of impingement may extend to both soft tissue impingement and bone impingement. Such detailed feedback may then be used to improve a proposed plan for implant placement. Additionally, these methods may be employed for several implant designs, such as tibial implant designs, either simultaneously or at different times. The designs may vary by size and/or type, and allow a user to compare expected performance between different implants to aid in the planning process.

In some embodiments, the planning method may also include selecting a size of a tibial implant. In particular, during the planning stage, once a kinematic pattern, such as a medial pivot pattern, is established, the kinematic pattern may be viewed overlaid on the tibial plateau. After virtually pinning the implant to a pivot point, e.g., pivot center based on average of convergence locations at different degrees of flexion, on the kinematic pattern, rotation of the tibial implant about the pivot point to fit a range of lateral maximum convergence points may not be possible, no matter the rotational position of the implant. In such circumstances, another virtual tibial implant may be retrieved from among a series of implants of different sizes and/or types, with the new implant replacing the original as pinned to the pivot point. This process of exchanging implants of different sizes and types may be repeated until an implant is selected that may satisfy kinematic requirements for position and orientation of the implant on the tibia while also maintaining geometric constraints of bony overhang and underhang. When this process is performed using software, a computer running the software may store any number or variety of implant sizes and types that may be retrieved using the software.

In some embodiments, the planning method may be preceded by an initial evaluation of whether a knee condition of the patient is healthy or otherwise as would be expected based on the patient's overall bone structure or whether it is damaged or diseased. This may be determined by performing a kinematic assessment on the patient. For instance, kinematic tests such as evaluation of motion in anterior-posterior, varus-valgus and interior-exterior degrees of freedom may be performed. In some instances, abnormal conditions may be evidenced by osteophytes, joint compartment collapse, or various degrees of impingement. If the knee is healthy, then the method proceeds as described above. If the knee is abnormal due to damage, disease or another condition, then the soft tissue in the knee joint may be evaluated more closely.

Assessment of an abnormal knee may proceed in various ways to arrive at a target kinematic pattern associated with a desirable post-operative outcome. In some examples, an assessment of the knee may include an evaluation of ligaments, tendons and cartilage, for example. The soft tissue is evaluated to identify each tissue that is abnormal relative to what would be expected in a healthy knee. Once all of the defective tissue is catalogued, various techniques may be employed to account for the abnormality in order to estimate the patient's kinematic pattern in a healthy or corrected condition. In this manner, a kinematic pattern based on a flexion range of motion in the existing condition of the knee may be modified to account for the abnormality. In some examples, accounting for the abnormality involves recreation of the soft tissue in a healthy state using a model to generate a predisease or preinjury state of the knee. Such model may include additional anatomy of the patient. This information may be utilized with the data collected for the relative movement of the tibia and femur through flexion range of motion to adjust the actual kinematic pattern to account for a healthy condition of the knee. In some examples, techniques other than the use of a model may be used to modify the kinematic pattern based on the specific condition of the abnormal soft tissue. In other examples, characteristics of the soft tissue may be used to extrapolate an expected kinematic pattern in a healthy state. Such characteristics may include actual attachment locations of the soft tissue and size or thickness of the soft tissue. To arrive at the expected healthy kinematic pattern, the measured kinematic pattern may be used as a baseline reference. A recreation of a pre-disease or otherwise corrected state may also be customized based on feedback from a patient prior to surgery. For example, if a patient complains of instability going down stairs and exhibits signs of anterior-posterior instability, a surgeon may impose specific constraints to account for such instability. In particular, a surgeon may prescribe no anterior-posterior movement in the medial condyle. Such constraint may also be incorporated into planning software for implant design.

In further embodiments, the evaluation of an initial condition of a knee may also involve circumstances where an apparent irregularity results from a discrepancy between individual kinematics of a patient and an implant design under consideration, rather than damage or disease. In such circumstances, the evaluation may prompt a change in a planned implant type to better suit the kinematics of the patient.

In still further embodiments, the initial assessment may reveal a damaged or otherwise abnormal knee where a kinematic pattern cannot be collected or is otherwise unreliable to the extent any pattern is identified. In such cases, the method may further provide for access to a set of pre-defined kinematic patterns from which the surgeon may select for use on the patient. For example, the set of pre-defined kinematic patterns may include one or more medial pivot, central pivot, lateral pivot and/or no pivot. The approach to selection from the set by a surgeon may be informed by various considerations that the surgeon may establish. For instance, the surgeon may obtain patient information based on patient activity prior to surgery or through a soft tissue laxity assessment in an intra-operative setting. In a variant, inputs regarding the patient may be processed by an algorithm to automatically select one or more pre-defined kinematic patterns from a set. Through the establishment of a kinematic pattern in the manner described, the surgeon has a target plan with which to reference for implant selection and alignment, as described in greater detail elsewhere in the present disclosure.

In some embodiments, a determination that a patient has abnormal kinematics such as the circumstances described above may prompt a warning to the user. Such warning may be accompanied by, for example, one or more of a kinematic pattern with or without an indication of its reliability, information regarding soft tissue characteristics, such as tension in ligaments or compromised ligaments, and information regarding impingement. Thus, the warning may provide an added layer of guidance in the planning process where abnormal kinematics are detected.

Returning to the planning for tibial implant design and alignment, in some embodiments, as described above, the kinematic pattern is viewed in the transverse plane overlaid on the tibial plateau such that a resection of the tibia is assumed to be perpendicular to a length of the tibia. In other embodiments, the method may benefit from a modification of the resection plane to improve positioning of a virtual tibial implant during the planning stage, and in these embodiments, a the tibial resection plan may be modified while viewing the kinematic pattern over a virtual tibial implant as part of the process of optimizing the implant position on the tibia. Such modification may be based on characteristics of one or more of anterior-posterior, medial-lateral or interior-exterior degrees of freedom through a range of motion in the joint. These characteristics are used for tibial planning including sagittal and transverse plane planning, though further modification of the resection plan may be possible based on optional gap balancing, described in greater detail elsewhere in the present disclosure. Consideration of the femur may also inform the knee planning steps, also described elsewhere in the present disclosure. Returning to planning based on the kinematic pattern of the tibia, in some examples, after pinning and orienting a virtual tibial implant, a result may be that the position of the tibial implant does not adequately accommodate the full kinematic pattern of the patient. At this juncture, the virtual resection cut of the tibia, initially presumed to be in the transverse plane, may be modified real time to bring the tibial implant into a more optimized position relative to the kinematic pattern. One way this may be done is through adjustment of an anterior-posterior slope of the tibial resection, though such example should not be considered limiting and other types of modifications may be made as deemed appropriate. The viewing of the kinematic pattern over the tibial plateau and over the tibial implant, along with adjustments to the planned tibial resection, may all be performed using software. Software may include tools to select from a catalogue of implants and various options to modify the resection plan. When the implant plan is viewed virtually, various conditions may be observed regarding an interface of the implants and their relationship to adjacent bone structures. For example, any impingement between implants may be visible, or gaps or overlaps between an implant and the kinematic pattern. In some examples, the virtual representation of the joint may be modified to account for expected characteristics of the knee joint that are evident based on the plan. This may involve, for example, modifying the implants to reduce or eliminate an expected impingement. Further, as described below, any adjustment to the resection plan based on the kinematic pattern may be taken into account if gap balancing is performed prior to actual trial or implant placement.

In some embodiments, planning for tibial implant placement as part of a larger scale knee surgery or replacement may also include performance of a gap balancing technique to ensure alignment of the femur and tibia in the coronal and sagittal plane. Ultimately, gap balancing involves checking to confirm that a rectangular flexion gap exists between the femur and the tibia in flexion and in extension. It may also involve checking to confirm that the gap is the same size in both flexion and extension. Moreover, gap balancing may be used to evaluate certain degrees of freedom of the tibia at the knee including superior-inferior, varus-valgus and flexion-extension, the results of which may inform the planning process, and in particular coronal plane planning. To the extent an executed planning method includes gap balancing, it should be appreciated that gap balancing may be utilized to obtain any desired subset of design parameters in order to complement the parameters obtained from kinematic data. In this manner, gap balancing may be used in any number of ways to provide additional information for planning.

This process is performed intraoperatively and as an initial step, the knee is accessed so that one of the tibia and the femur may be resected. Soft tissue releases are then performed to balance the joint or implant positions are modified to minimize this need. When the gap in the first position is confirmed, resection of the other bone is performed to begin the remainder of the gap balancing process. Completion of gap balancing may allow for the positioning of a femoral implant, if used, and may, in some cases, lead to a refinement of a position of the tibial implant. The aforementioned steps of the gap balancing process may be modified as known in the art. Further, additional details of the gap balancing process will be known to persons of ordinary skill and may be utilized to achieve the purposes of gap balancing as discussed herein.

In some cases, resection of one or both of the tibia and femur may be modified to achieve gap balancing. This may involve, for example, modifying a surface angle or depth of resection. Further, any modified resection contemplated based on planning for the tibial implant position and orientation may also be accounted for during this process. In some examples, gap balancing may be performed after virtual determination of a tibial implant placement including its position and orientation relative to the tibia. In other examples, gap balancing may be performed after the kinematic pattern is obtained but prior to a final determination of the tibial implant position and orientation. As mentioned above, gap balancing may be used for coronal plane planning to complement planning based on a kinematic pattern. Nonetheless, it should be appreciated that some embodiments of the method will not involve or require any gap balancing.

In another aspect, the present disclosure relates to a method of planning a position of a femoral implant on a knee joint, planning for selection of such femoral implant or planning the position and the selection of such femoral implant. In one embodiment, the method includes a step of performing a range of motion of the tibia relative to the femur while capturing data on the relative positions of the tibia and the femur at increments through the range of motion. The data may be collected through techniques as described above for the tibial implant planning method. In some examples, this may be a technique not requiring x-rays or fiducial markers, such as visual imaging based motion capture. In other examples, a surgeon may perform a dynamic passive flexion test to obtain the data. And, as with the tibial implant planning method, such data may be collected pre-operatively or intra-operatively.

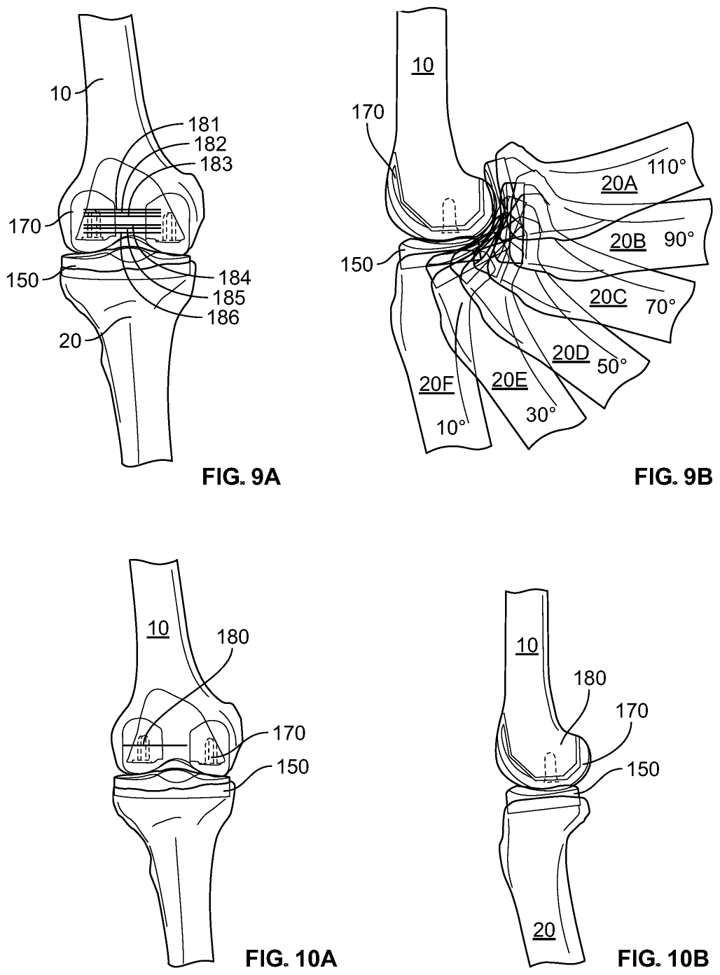

The data collected based on the range of motion is analyzed to determine a dynamic flexion axis through the femur, also known as a flexion helical axis, at different flexion angles through the range of motion. The dynamic flexion axis is a center of rotation of the tibia relative to the femur where the rotation is in the sagittal plane. Thus, when viewed in the sagittal plane, the dynamic flexion axis appears as resembling a dot because it is generally perpendicular to the sagittal plane. The dynamic flexion axis may provide information to evaluate anterior-posterior, superior-inferior, interior-exterior and varus-valgus degrees of freedom to plan for femoral implant design and alignment, e.g., alignment in sagittal, transverse, and coronal planes. The data representative of the dynamic flexion axis may be collected by and stored in a computer storage device. Further, software may be operated to view the data in text or visual form on a user interface. The knee joint may be viewed from different positions, such as in a coronal plane, as shown in FIG. 9A or a sagittal plane as shown in FIG. 9B. The software may also display virtual implants overlaid on applicable bone surfaces shown on the user interface. In FIGS. 9A and 9B, tibial implant 150 and femoral implant 170 are shown overlaid on patient anatomy. The software may also simultaneously display the joint at different flexion angles over range of motion 7 that include data for each angle collected or for a subset of angles. In FIG. 9B, positions of tibia 20A-20F relative to femur 10 are shown at 10, 30, 50, 70, 90 and 110 degrees of flexion, with corresponding dynamic flexion axes also shown.

The data collected by the computer regarding the dynamic flexion axis at different flexion angles, such as dynamic flexion axes 181-186 shown in FIG. 9A, is processed to determine a femoral knee center axis 180, as shown in FIGS. 10A-10B. This determination may involve a calculation of an average position of the dynamic flexion axis based on the dynamic flexion axes at each available flexion angle. In some examples, a subset of the dynamic flexion axes may be used to determine the average. This may be desirable where data regarding the dynamic flexion axis at a particular flexion angle is well outside of an expected position relative to the other data points. In further examples, a formula other than an average of the combined dynamic flexion axes may be used.

With a femoral knee center axis 180 established, a planned implanted position of the femoral implant may be determined and optimized. Axis 180 may be viewed in the coronal (FIG. 10A), sagittal (FIG. 10B) and transverse (not shown) planes, and a position of the femoral implant may be adjusted virtually on a user interface display. Thus, an angle of femoral implant 170 relative to a longitudinal axis of the femur in the coronal plane may be adjusted, a rotational position of femoral implant 170 in the sagittal plane may be adjusted based on axis 180, and a rotation of the femoral implant in the transverse plane may also be adjusted in view of axis 180. Through these adjustments, the virtual femoral implant may be aligned with the femoral knee center axis. Femoral knee center axis 180 may also be used to optimize a size of a femoral implant. The determination of the planned implanted position of the femoral implant may be accomplished in many ways. For instance, a surgeon may view the femoral knee center axis 180 from various angles and use judgment to position femoral implant 170. In other examples, software may be used to execute an algorithm to automatically determine an optimal implant position based on the anatomy, femoral knee center axis 180, and the virtual femoral implant shape. In still further examples, a hybrid approach may be used where the surgeon may make adjustments to a plan automatically generated by software.

In some examples, the data collected through the performance of range of motion 7 may be used to select a femoral implant type and/or size. Selection may be based on analysis of a virtual implant relative to femoral knee center axis 180 from different directions, such as in the coronal, sagittal or transverse plane. The selection process may be performed in the ways described above, including by a surgeon, automatically with software, or through a combination of both. In still further examples, a method may be performed that involves both implant position and selection determinations.

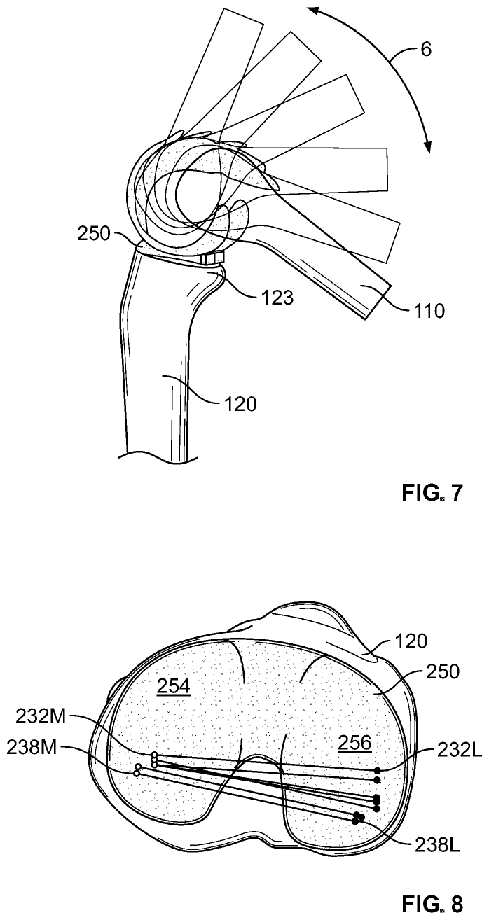

Another aspect of the present disclosure relates to a method of planning an implant placement using a virtual implant and evaluating the implant placement with physical implantation of a trial or implant. In one embodiment, the method commences with planning a tibial implant position and orientation, as described above and shown in FIGS. 1 and 4-8. The planning may be performed virtually. Once a plan is established, the knee of the patient is accessed to prepare the necessary resections in furtherance of the surgical plan. Once the site is prepared, a trial tibial implant or tibial implant 250 is positioned according to the plan on the resected proximal tibial surface 23. With the implant set in position, the joint may once again be moved through a range of motion 6, as shown in FIG. 7. Through this process, a second kinematic pattern through the flexion range of motion may be procured based on the kinematics of the joint with the tibial implant 250 positioned therein. Continuing with the example of the preceding embodiment, the second kinematic pattern established in FIG. 8 continues to be a medial pivot with medial maximum convergence points 232M-238M and lateral maximum convergence points 232L-238L. The second kinematic pattern may then be compared against the articular surfaces 254, 256 of implant 250 to evaluate whether the implant is performing as expected based on the original surgical plan. This allows for modifications of the implant position in the event there is any discrepancy between the performance with the trial or implant 250, and the performance predicted through the earlier established plan.

In some embodiments, the method including implantation of a trial or implant may also include a gap balancing procedure prior to initial placement of the trial or implant. The gap balancing may be performed as described elsewhere in the present disclosure.

Additional aspects of the present disclosure relate to instruments, systems and methods of use thereof for selecting trials or implants for use in a mammalian joint and positioning such trials or implants in the joint.

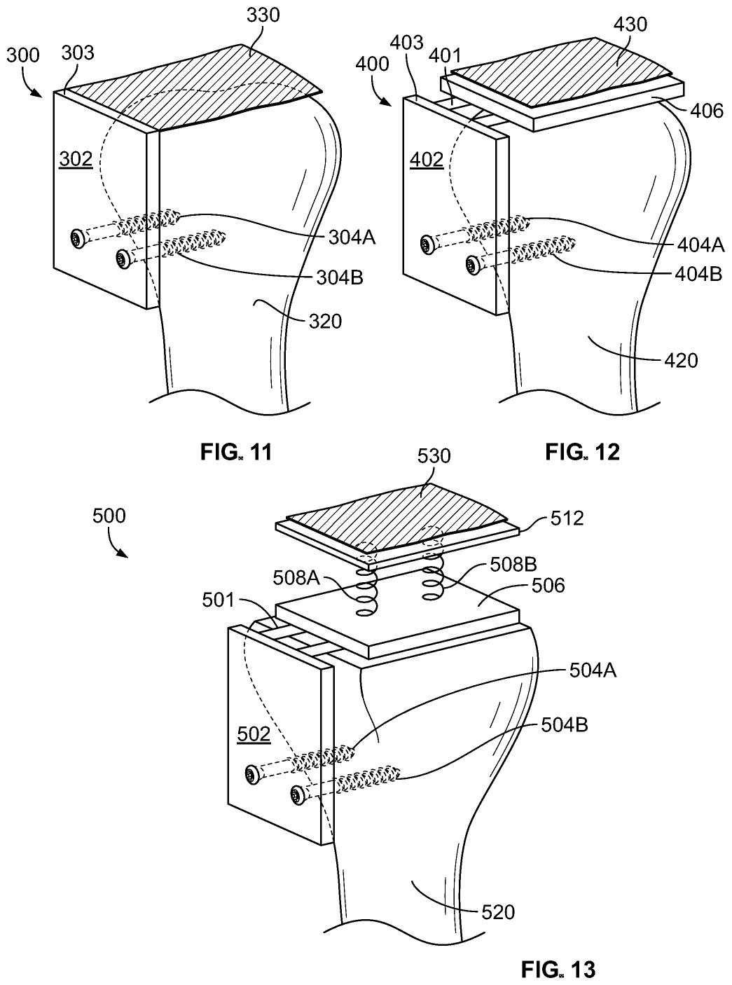

In one aspect, the present disclosure relates to a positioning instrument. Certain embodiments of the positioning instrument are shown in FIGS. 11-13. In one embodiment, positioning instrument 300 is shown in FIG. 11. Positioning instrument 300 includes a body 302 with an upper surface 303. Positioning instrument 300 may be made of sterilizable material such as stainless steel or a thermoplastic. A sheet 330 is attached to the body at the upper surface 303. It should be appreciated that any form of attachment may be used to secure sheet 330 to body 302, and that sheet 330 may be connected to the body at locations other than upper surface 303. Sheet 330 may be sized so that when sheet 330 is extended over an end of a tibia 320, the sheet is long enough to cover the tibial surface, as shown in FIG. 11.

With continued reference to sheet 330, the sheet may also be known in some examples as articulation paper. In some examples, the sheet may be paper or a sensor film. One type of sensor film that may be used is a polymeric film having pressure-sensing characteristics in the form of microcapsules. When pressure is applied to the microcapsules, they rupture and cause a colored ink or dye to be absorbed within the film, thereby leaving visible markings on the film. One example of this type of sensor film is Fujifilm® Prescale pressure measurement film. In another type of pressure-sensitive sheet, sheet 330 may be a polymer or other material with particular physical characteristics such that a surface of the material changes in appearance upon the application of pressure. One way a polymer material may be prepared to serve this function is through embedding nanoparticles in the polymer. When the nanoparticles spread further apart under pressure, an appearance of the regions subject to pressure changes relative to the regions not subject to pressure. In other examples, sheet 330 may incorporate other sensor technology. Turning to specific examples, in one example, the sheet may be a biocompatible paper with sufficient tear resistance to withstand tension due to a bone surface rotating on the paper in an intraoperative setting. In this example, the sheet may be complemented by a biocompatible dye applied to the condyles of the femur so that a path of contact between the femur and the tibia is imprinted on the sheet through a rotation of the joint through a range of motion.

In yet another example, sheet 330 incorporates capacitive sensing technology that relies on an electrostatic field. Because the present disclosure seeks to capture, in relevant part, contact between a femur and a tibia at two separate locations, i.e., medial condyle and lateral condyle, mutual capacitance may be used. In this example, sheet 330 includes sensors embedded therein to detect locations of pressure based on contact between the femur and the tibia over a range of motion. For instance, a metal alloy such as indium tin oxide (ITO) or copper material may be used as a material to define a grid in a plane parallel to the sheet surface. A first series of parallel strands of the alloy may define a first layer and a second series of parallel strands perpendicular to the first series may define a second layer separated from the first by an insulator. These layers may in turn be overlaid on a support base and may be connected to a controller to collect data from the sensors. The controller may include a processor and memory and other components that facilitate and optimize the processing, storage and transmission of data collected from the sensors. The controller may be physically attached to or embedded within the instrument body or it may be located remotely. Wireless technology may be incorporated into the instrument for communication with outside computing devices and, where applicable, for communication with a remote controller associated with the sheet.

Positioning instrument 300 also includes anchors 304A-B attached to body 302. The anchors may be bolts or other known securement mechanisms. Anchorage for the positioning instrument may include a single anchor or three or more anchors. The anchor or anchors are sized and comprise materials suitable for anchorage of body 302 into a bone to hold positioning instrument 300 in a fixed position relative to the bone. In some examples, positioning instrument 300 may also include additional clips, pins or other similar fastening mechanisms (not shown) to hold sheet 330 in place on tibia 320 once sheet 330 is laid over the tibial surface.

In another embodiment, positioning instrument 400 is shown in FIG. 12. In FIG. 12, reference to the 400 series of numerals refers to like elements in the 300 series of numerals unless otherwise stated. Positioning instrument 400 includes a body 402 and a platform 406. Body 402 is secured to a tibia via anchors 404A-B. Platform 406 may be directly connected to body 402 or may be connected to a bridging structure 401 between the platform and the body, as shown in FIG. 12. Bridging structure 401 may be rigid or may have resilient properties to allow some movement between platform 406 and base 402. In some variations, platform 406 may be a rigid construct such that its shape remains constant even when not disposed on a supporting surface while bridging structure 401 is resilient. In other variations, platform 406 may have resilient properties while bridging structure is either rigid or resilient. Body 402 and platform 406 are arranged such that a joint facing surface of the platform is transverse to an elongate direction of body 402, as shown in FIG. 12. Positioning instrument 400 is also complemented by sheet 430, which may be secured directly to platform 406 in a manner such that sheet 430 is spaced apart from body 402, as shown in FIG. 12. Alternatively, sheet 430 may extend directly from body 402. In some examples, positioning instrument 400 may also include additional clips, pins or other similar fastening mechanisms (not shown) to hold sheet 430 in place relative to platform 406.

In another embodiment, positioning instrument 500 is shown in FIG. 13. In FIG. 13, reference to the 500 series of numerals refers to like elements in the 300 series of numerals unless otherwise stated. As with the preceding embodiments, positioning instrument 500 includes a body 502 secured in place on a tibia 520 with anchors 504A-B. Instrument 500 also includes a lower platform 506 attached to body 502, and an upper platform 512 directly above lower platform 506 and secured thereto via a resilient member. Lower platform 506 may be directly attached to body 502 or may be connected to a bridging structure 501, as shown in FIG. 13. Bridging structure may be rigid or may have resilient properties to allow some movement between platform 506 and base 502. Such resilience may provide a range for positioning lower platform 506 relative to the body based on the position of body 502 when anchored to the tibia.

In FIG. 13, the resilient member in between platforms 506, 512 is a pair of coil springs 508A, 508B, though it is contemplated that other structures with elastic properties may be used as the resilient member. Secured or otherwise positioned on upper platform 512 is sheet 530. In some examples, a single resilient member or three or more resilient members may be used. In some examples, lower platform 506 and upper platform 512 have approximately the same footprint. And, in some examples, positioning instrument 500 may also include additional clips, pins or other similar fastening mechanisms (not shown) to hold sheet 530 in place relative to platform 512.

The positioning instrument may be varied in many ways. In some examples, including variations of the embodiments shown in FIGS. 11-13, the base may include a built-in slot or other similar feature so that when the base is anchored to a bone, a position of the base relative to the bone may be adjusted to optimize a location of the sheet over the bone. Still further examples may include a locking mechanism to hold the body in place relative to the anchorage at a desired position. In other examples of any one of the above embodiments, including variations of those shown in FIGS. 11-13, the instrument may be a platform with a handle extending therefrom, with a sheet attachable to the platform. In these examples, the handle takes the place of the body and the anchorage, and the instrument is fully portable. The instrument of these examples is adapted so that the instrument may be held and brought into position over the tibia while being held. The instrument platform may be equipped with pins or other attachment features to hold the platform in place on the tibia prior to commencing a range of motion procedure.

In another aspect, the present disclosure relates to a kit including one or more positioning instruments and one or more tibial trials and/or implants. In some examples, one of the tibial trials or implants of the kit may be translucent or transparent so that locations of the medial and lateral paths of contact are visible on the sheet with the trial disposed thereon. In other examples, the tibial trial may include bores at the medial and lateral sulcus so that a surface below the trial is visible from the top, the surface being in alignment with the sulcus. In some examples, the trial may be translucent. In other examples, the trial may be opaque or have any degree of translucency while also including visualization bores.

In another aspect, the positioning instrument of any one of the embodiments of the present disclosure may form part of a positioning system that includes a computer and one or more navigation tools. In some examples, the system may include a positioning instrument, a computer, a probe instrument, fiducial markers for attachment to one or more of the probe instrument, the tibia and the trial, a camera, such as a stereoscopic camera, a display associated with the computer, and software to process data flows associated with navigation data collected from the probe and the operated upon anatomy. With these tools, locations of the tibia and the trial may be monitored real-time in the intra-operative setting. In some examples, the probe instrument may be used to collect coordinates of anatomical locations at the surgical site. This may be to collect points on the tibia to register the tibia with the system or it may be to collect additional points during surgery. For instance, the probe may collect locations marked on the sheet over the tibia, where the markings are representative of the established medial path of contact and the lateral path of contact. Once collected, such points may be generated on the display including a virtual model of the tibia. Similarly, the system may also be adapted to register one or more trials or implants so a location of the trial or implant may be monitored real-time.

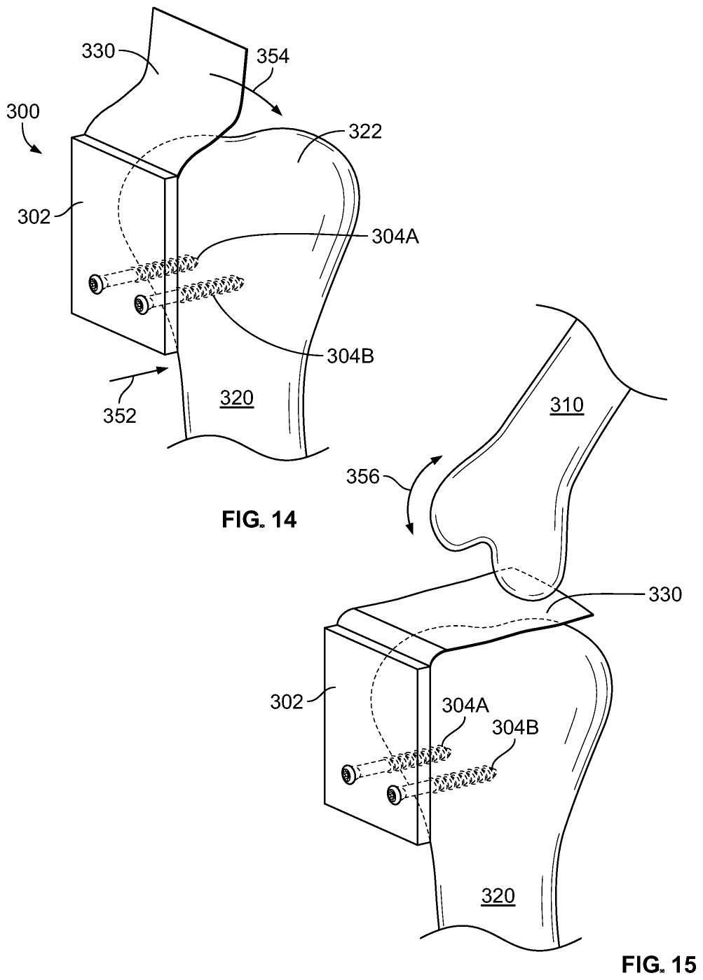

In another aspect, the present disclosure relates to a method of positioning a trial or an implant on an end of a bone facing a joint, such as a proximal end of a tibia. One embodiment of the method is illustrated in FIGS. 14-17. For purposes of illustration, positioning instrument 300 is referenced for performance of the method, though it should be appreciated that the method is not limited to implementation with instrument 300 and may be carried out using other instruments as contemplated by the present disclosure.

With access to a knee of a patient prepared, instrument 300 is brought toward the surgical site intended to receive a tibial implant. Once a determination is made as to a position of the instrument that will allow for adequate coverage of tibia 320 by sheet 330, anchors 304A-B are advanced into tibia 320 to anchor instrument 300 in place, as indicated by reference numeral 352. Sheet 330 is then unfurled as necessary to position its surface over an end surface 322 of tibia 320, as shown in FIG. 14 and indicated by reference numeral 354. Optionally, pins or other small anchorage elements (not shown) may be included on an outer periphery of the sheet to hold the sheet against the tibial bone surface. Where instrument 400 is used, sheet 430 may be optionally secured to platform 406. Where supplemental securement is used to hold the sheet in place, such securement is temporary and may be removed upon completion of the procedure to determine medial and lateral path centers.

Once the sheet 330 is deemed to be satisfactorily positioned on the tibia, tibia 320 may be rotated relative to femur 310 to move the knee joint through a range of motion with sheet 330 in position on the tibia, as shown in FIG. 15 and indicated by reference numeral 356. The range of motion should be at least a significant portion of an overall range of motion of the patient to best capture the relationship of the tibia and the femur throughout the range. As the tibia is rotated, contact points between the lateral side of the tibia and the lateral condyle of the femur and between the medial side of the tibia and the medial condyle of the femur are collected by the sheet. The number of contact locations collected over the course of the rotational movement may be a quantity based on predetermined intervals of time or distance, random intervals of time or distance, or may be based on continuous collection. This method step is the same irrespective of the type of sheet used and irrespective of whether physical markings or other sensors such as sensors based on pressure sensitive films are relied on for the paths of contact between the bones. Nevertheless, the manner in which the collected contact locations are used may vary between sheets with a physical representation of the contact locations, i.e., markings visible on the sheet, and sheets where the contact locations are collected digitally, e.g., with capacitive sensors.

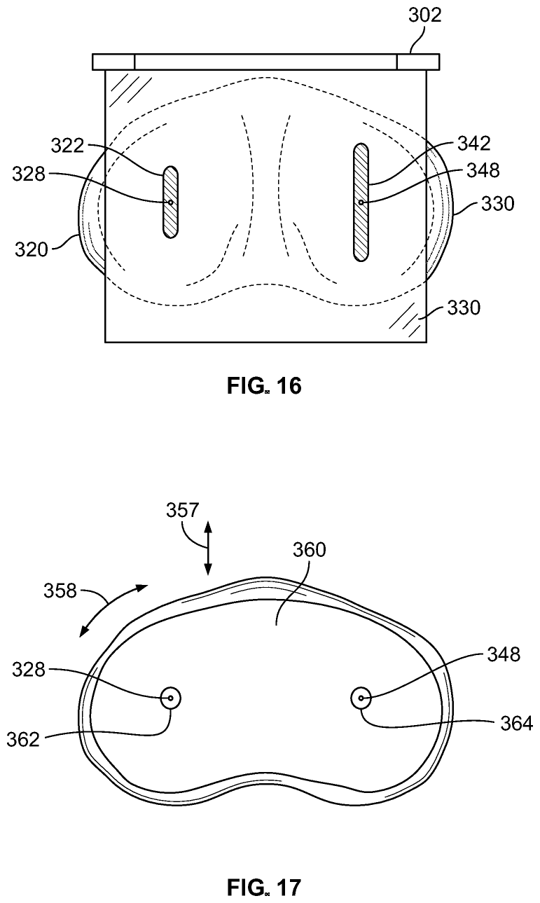

Upon completion of the rotation through the range of motion, the sheet has recorded or otherwise collected a medial path of contact 322 and a lateral path of contact 342 between the tibia and the femur, as shown in FIG. 16. A range of medial path of contact 322 is used to calculate a medial path center 328 and a range of lateral path of contact 342 is used to calculate a lateral path center 348. Each path center is determined based on a geometric center of the contact points. Preferably, a near full range of motion is performed to obtain the ranges of medial and lateral paths of contact. To the extent a shorter range of motion is used, care should be taken to ensure that a respective degree of extension and flexion at the ends of the range is similar.

Optionally, the medial path of contact and the lateral path of contact may be used to evaluate the type of kinematic pattern exhibited by the patient, such as whether the pattern is a medial-pivot or a lateral-pivot. A boundary between these different types of patterns may be adjusted through a surgeon-selected coefficient. A simple ratio may be used to determine the pattern. For the ratio, a length of medial path of contact 322 may be represented by DM, a length of lateral path of contact 342 may be represented by DL, and the surgeon selected coefficient may be represented by a* having a value from 1.0 to 2.0. The applicable ratio is then assessed as follows:

D_{L}≥D_{M}×a^{*}(medial stability designs/medial pivot)

D_{L}<D_{M}×a^{*}(symmetrical designs/lateral pivot)

Determination of the applicable type of kinematic pattern allows a surgeon to make a threshold determination of what type of trial to select for placement before beginning the process of positioning a trial on the tibia. For instance, based on the kinematic pattern, a trial with a medial-pivot may be preferred over a trial with a lateral-pivot. Optionally, in subsequent steps of the method, an implant may also be retrieved in place of a trial.

With this patient-specific information now determined and applied on the tibia of the patient in the intra-operative setting, a tibial trial 360 may be retrieved and positioned so that a medial sulcus of the trial is aligned with medial path center 328 and a lateral sulcus of the trial is aligned with lateral path center 348. Initial selection of the trial may be based on past experience of the surgeon, the patient-specific kinematic pattern, or a combination of both. Alignment may involve translation 357 and rotation 358 of trial 360, as shown in FIG. 17.

In some examples, the trial is translucent or transparent, and locations of the path centers 328, 348 are visible through the trial as its position on the tibia is adjusted. In other examples, such as with trial 360 shown in FIG. 17, the trial includes bores 362, 364 aligned with the respective medial and lateral sulcus of the trial. Such bores 362, 364 pass from a joint-facing surface of trial 360 to a tibia-facing surface of the trial. In this manner, when the trial 360 is positioned on the sheet 330, which in turn is overlaid on the tibia, the surface of the sheet is visible through the bores of trial 360. And, because the medial and lateral path centers are physically visible on the sheet, the bores of the trial provide a visual aid to align the trial with the path centers. For instance, the trial 360 may be positioned so that the path centers 328, 348 on the sheet are visible through the bores 362, 364, where such position represents a desired position for the trial. These method steps ensure that a trial is positioned on the tibia to conform to the patient-specific kinematics of the patient at issue and accordingly improves the optimization of implant positioning. Once the trial is deemed to be satisfactorily aligned, the positioning instrument and sheet may be removed. Further, the trial may be pinned or otherwise temporarily secured in place at the desired position on the tibia for further evaluation.

In some variations of the above embodiment, the method may also optionally be used to determine an implant size. For instance, if after positioning trial 360 as shown in FIG. 17 it is determined that the trial does not adequately fit over the tibia, then a trial with a different size or design may be retrieved and the trialing process may thereafter be repeated with the new trial.

The method shown in FIGS. 14-17 may be varied in many ways in addition to those stated above. In one example, the tibia may be resected prior to attachment of the positioning instrument to the tibia. In such instances, positioning instrument 300 or 400 may be used, though depending on the depth of resection, use of instrument 500, described below, may be preferred. In other examples, resection of the tibia may be performed after attachment and use of the positioning instrument, but before the placement of a trial or implant on the tibia.

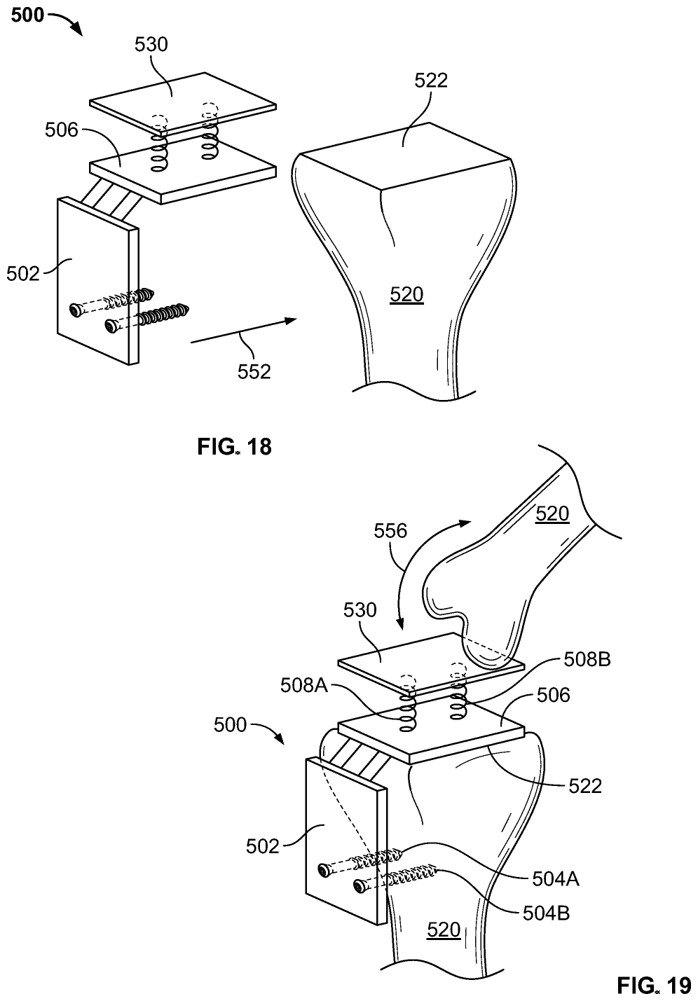

FIGS. 18-21 illustrate another embodiment of a method of using a positioning instrument to position a tibial trial or implant on a tibia. In this embodiment, the method begins after surgical access to the knee is prepared and a proximal end of tibia 520 is resected leaving resected end surface 522, as shown in FIG. 18. Instrument 500 is then advanced toward the surgical site and anchored into tibia 520, advancement being indicated by reference numeral 552 in FIG. 18. As with other embodiments of the present disclosure, an anchorage location is chosen so that sheet 530 mostly or fully covers a footprint of resected end surface 522. Although tibia 520 is resected, sheet 530 is elevated relative to lower platform 506 when no loads are applied to upper platform 512, as shown in FIG. 19. Such position of sheet 530 results from resilient member 508A-B being expanded in an unbiased position, resilient member 508A-B connecting lower platform 506 to upper platform 512 with sheet 530 thereon. With sheet 530 elevated, an otherwise remote surface of the resected tibia is raised so that rotation of the knee joint will bring about contact between femur 510 and sheet 530. It should be appreciated that the sheet used in the method has physical properties to withstand expected forces from movement of the bones in the knee joint. Additionally, the presence of the resilient element allows for sufficient additional movement of the bones in the joint to reduce any risk of damage to surrounding soft tissue.

With positioning instrument 500 being in a desired anchorage position, the tibia is rotated relative to the femur and placed through a range of motion to capture both a medial path of contact and a lateral path of contact between the bones. The resultant outputs are the same as those shown in FIG. 16, though in this instance contact points are collected on sheet 530, not sheet 330. With the contact points collected, a medial path center is determined based on the points representing the medial path of contact and a lateral path center is determined based on the points representing the lateral path of contact. The knee is then ready for evaluation of a trial.

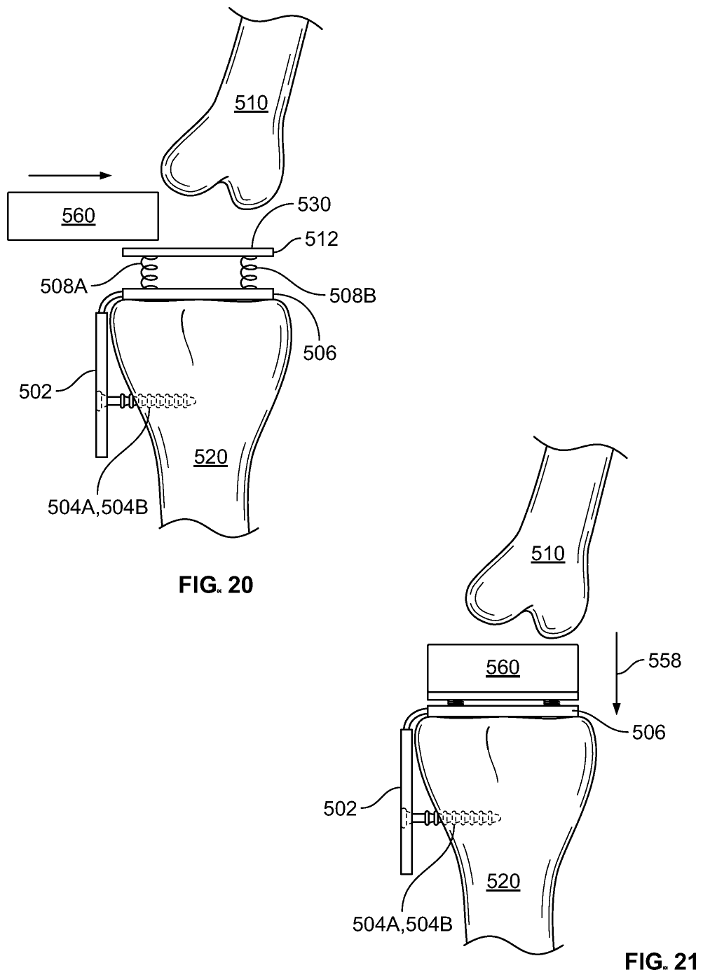

FIGS. 20-21 illustrate placement of trial 560 on sheet 530 overlying upper platform 512. It should be recognized that instrument 500 is designed so that when trial 560 is placed on upper platform 512, upper platform 512 is pressed against lower platform 506 while resilient member 508A-B is compressed, as shown by the change between FIGS. 20 and 21 and indicated by reference numeral 558. A position of tibial trial 560 may then be adjusted through translation and/or rotation to align a medial sulcus (not shown) of trial 560 with a medial path center and a lateral sulcus (not shown) of trial 560 with a lateral path center, as described in greater detail for the method illustrated in FIGS. 14-17. And, not only does positioning instrument 500 facilitate performance of a range of motion procedure on resected tibial surface 522, the knee joint may also be rotated with the trial in place on the resected surface to evaluate the kinematic fit and to confirm the suitability of the chosen position for the trial.

The methods of embodiments including those illustrated in FIGS. 14-17 and 18-21 may be varied in many ways. In some examples, the described method may be complemented by the use of a system including a computer and various tools to assist with navigation, particularly for the purpose of positioning the trial on the tibia. Prior to evaluating the medial and lateral paths of contact, the tibia may be registered on the system so that its coordinates may be observed in real time through a virtual model. To register the tibia, one or more fiducial markers are placed on the tibia, a stereoscopic camera is positioned in a field of view of the tibia, then a previously registered probe instrument is brought into the surgical field to collect points on the tibia to register it with the software of the computer. Once the medial and lateral paths of contact are determined, the probe instrument may be used to record those contact points so they may be transposed onto the virtual model of the tibia. The virtual model of the tibia and additional data such as the medial and lateral paths of contact may be viewed on a display connected to the computer. Once coordinates for the medial and lateral paths of contact are collected, the computer may calculate the medial and lateral path centers and also display those in the virtual model. The computer may be configured so that such calculation is automatic when the data is received, or the computer may be configured so that the calculation occurs when prompted by a user of the system. A tibial trial or implant that is ready to be introduced to the surgical site may be registered as well so that its real-time location is visible on a display in the same field as the virtual model. This may be done by attaching a fiducial marker to the trial and then moving the trial while in view of the camera. Then, when the registered trial is positioned on the registered tibia, the displayed virtual model may be used to visualize alignment of the medial and lateral sulcus of the trial with the respective medial and lateral path centers 328, 348, to optimize a position of the trial.

In some examples, the sheet used may incorporate sensors, as noted in the description of the method above and in the description of the positioning instrument. In instances where the sensors do not induce a physical manifestation of contact locations on the sheet itself, such as when capacitive sensors are included in the sheet, the sensors transmit a signal to a controller regarding the coordinates of contact locations along the range of motion of the joint. The controller, in turn, is operatively connected to the computer and navigation tools used to generate a real-time virtual model of the tibia, as described in the previous example. In this way, the computer may generate and display an output of the contact locations on the virtual model of the tibia in real time. Additionally, the computer may, either automatically or upon being prompted, calculate medial and lateral path centers 328, 348 based on the collective contact points, i.e., medial and lateral paths of contact 322, 342, received from the sensors. The computer may also display such medial and lateral path centers on the virtual model. Then, as a registered trial or implant is brought into the surgical site to be placed on the tibia, the medial and lateral sulcus of the trial may be lined up to the path centers on the tibia by physically adjusting the trial position while simultaneously observing the alignment of the sulcus points and tibial surface path centers on the virtual model, as also described in the previous example.

In any of the contemplated embodiments and examples thereof, the method may continue after positioning of an initial trial in the event that the initial trial or implant is determined not to fit on the tibia. For instance, this may include a circumstance where after alignment of the trial with the medial and lateral path centers on the tibia, the trial overhangs an outer edge of the tibia. In such cases, a second trial or implant may be retrieved and aligned on the tibia following the same procedure. Where the initial trial was positioned with the aid of a computer and navigation, such supporting components may continue to be used to align the second trial.

In any of the contemplated embodiments and examples thereof, the method may proceed from tibial trial positioning on the tibia by pinning the trial in the desired rotational position on the tibia, followed by removal of the positioning instrument. Then, the tibial trial may be pinned at a second location to secure its rotational position relative to the tibia.

The present disclosure is advantageous in that it facilitates improved tibial implant selection and, for any given selection, improved rotational and translational positioning on the tibia. In particular, alignment of a tibial implant based on the patient-specific kinematic pattern of the patient leverages the use of the patient's natural contact centers on the medial and lateral sides of the knee.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A method of planning implantation of a knee prosthesis in a patient comprising:
in response to rotation of a tibia of the patient through a range of motion relative to a femur of the patient, the range of motion including at least part of a distance between extension of the knee and flexion of the knee, collecting data representative of a position of the tibia relative to the femur at a plurality of orientations of the tibia relative to the femur such that data is collected for a plurality of positions, the data at each position of the plurality of positions including:
a medial contact location defined by a maximum convergence of a low point on a medial condyle of the femur and a medial tibial articular surface of the tibia; and
a lateral contact location defined by a maximum convergence of a low point on a lateral condyle of the femur and a lateral tibial articular surface of the tibia;
analyzing the medial and lateral contact locations from the plurality of positions to determine a kinematic pattern; and
utilizing the kinematic pattern to determine an implant position and an implant orientation for a tibial implant to be placed on a resected tibial surface.

2. The method of claim 1, wherein the kinematic pattern is in a transverse plane and includes a plurality of lateral contact locations and a plurality of medial contact locations, both inclusive of the plurality of positions, the plurality of lateral contact locations defining a lengthwise sequence along the lateral tibial articular surface, and the plurality of medial contact locations collectively defining a pivot point on the medial tibial articular surface such that the tibia rotates about the pivot point as the knee joint moves between extension and flexion.

3. The method of claim 2, wherein utilizing the kinematic pattern to determine the implant position and the implant orientation for the tibial implant involves centering a medial sulcus point of a medial articular surface of the tibial implant on the pivot point and then rotating the tibial implant about the pivot point such that the plurality of lateral contact locations are all interior to a perimeter of a lateral articular surface of the tibial implant.

4. The method of claim 1, further comprising selecting at least one of a size of the tibial implant and surface characteristics of the tibial implant based on the kinematic pattern.

5. The method of claim 1, further comprising modifying a planned resection cut of the tibia such that the planned resection cut is not entirely flush with a transverse plane, the modification improving correspondence between the kinematic pattern and the tibial implant when the kinematic pattern is overlaid on the tibial implant.

6. The method of claim 1, further comprising performing gap balancing of the knee joint at different orientations of the tibia relative to the femur to determine a first cut location on the tibia, the first cut location defining a proximal tibial surface for receipt of the tibial implant.

7. The method of claim 1, wherein the kinematic pattern is in a transverse plane and includes a first range of medial contact locations and a second range of lateral contact locations the first range and the second range being separated by a pivot point on a first location on the tibia such that the tibia rotates about the first location as the knee joint moves between extension and flexion, the first location being identifiable relative to an anatomical reference on the tibia.

8. The method of claim 7, wherein utilizing the kinematic pattern to determine the implant position and the implant orientation for the tibial implant involves centering the tibial implant on the first location of the tibia and then rotating the tibial implant such that a perimeter of a medial articular surface of the tibial implant envelopes all medial contact locations of the kinematic pattern and a perimeter of a lateral articular surface of the tibial implant envelopes all lateral contact locations of the kinematic pattern.

9. The method of claim 1, wherein the collecting of data is performed with a tracking device to monitor the position of the tibia and the femur throughout the range of motion.

10. The method of claim 1, wherein collecting data further comprises collecting data at each position of the plurality of positions with a sheet positioned on the tibia such that the medial and lateral contact locations are recorded by sensors on the sheet, the sheet being part of an instrument secured to the tibia.

11. A method of implanting a knee prosthesis comprising:
the method of planning implantation of the knee prosthesis according to claim 1;
resecting the tibia to prepare the resected tibial surface;
obtaining the tibial implant; and
positioning the tibial implant on the resected tibial surface according to the determined position and orientation of the tibial implant.

12. The method of claim 1, wherein utilizing the kinematic pattern to determine the implant position and the implant orientation is accomplished with the use of a virtual implant overlaid on both a virtual representation of the kinematic pattern and a virtual model of the tibia.

13. A method of planning an implant placement in a patient comprising:
retrieving data at a plurality of positions of a tibia of the patient relative to a femur of the patient, the plurality of positions collectively representative of at least part of a range of motion of a knee of the patient, wherein the data at each of the plurality of positions includes:
a medial contact location defined by a maximum convergence of a low point on a medial condyle of the femur and a first medial tibial articular surface of the tibia; and
a lateral contact location defined by a maximum convergence of a low point on a lateral condyle of the femur and a first lateral tibial articular surface of the tibia;
analyzing the data collected from the plurality of positions to determine a range of medial contact locations based on the at least part of the range of motion and a range of lateral contact locations based on the at least part of the range of motion;
virtually selecting a virtual tibial implant with a second medial tibial articular surface and a second lateral tibial articular surface; and
determining a planned implant position for the virtual tibial implant and a planned implant orientation for the virtual tibial implant by positioning and orienting the virtual tibial implant such that the range of medial contact locations are overlaid within the second medial tibial articular surface and the range of lateral contact locations are overlaid within the second lateral tibial articular surface.

14. The method of claim 13, wherein the range of lateral contact locations collectively defines a first lengthwise sequence in a transverse plane and the range of medial contact locations collectively defines a second lengthwise sequence in a transverse plane, the first and second lengthwise sequences together defining a pivot point on one of the range of lateral contact locations, the range of medial contact locations and an intercondylar eminence, the implant position being determined in part by the virtual tibial implant being positioned so that an anatomical feature on the virtual tibial implant that corresponds to an anatomical feature at the pivot point is aligned with the anatomical feature at the pivot point.

15. The method of claim 14, wherein determining the planned implant position of the virtual tibial implant involves centering a medial sulcus point of a medial articular surface of the virtual tibial implant on the pivot point, the pivot point being on the range of medial contact locations.

16. The method of claim 14, wherein determining the planned implant orientation of the virtual tibial implant involves rotating the virtual tibial implant about the pivot point such that the range of lateral contact locations are overlaid within the second lateral tibial articular surface and the range of medial contact locations are overlaid within the second medial tibial articular surface.

17. A method of implanting a total knee prosthesis in a patient comprising:
the method of planning according to claim 13;
resecting the tibia to define a resected tibial surface; and
placing a tibial implant corresponding to the virtual tibial implant on the resected tibial surface according to the planned implant position and the planned implant orientation.

18. The method of claim 17, further comprising:
retrieving implant data at a second plurality of positions of the tibia relative to the femur with the tibial implant positioned on the resected tibial surface, the second plurality of positions collectively representative of at least part of the range of motion, wherein the data at each of the plurality of positions includes the medial contact location and the lateral contact location with the tibial implant in place on the tibia;
analyzing the implant data collected from the second plurality of positions to determine a second kinematic pattern based on a maximum convergence of the tibial implant and the femur at the second plurality of positions; and
comparing the second kinematic pattern based on the implant data with a first kinematic pattern including the range of medial contact locations and the range of lateral contact locations.

19. The method of claim 18, further comprising changing at least one of the planned implant position and the planned implant orientation when a difference between the second kinematic pattern and the first kinematic pattern is above a predetermined threshold.

20. A method of evaluating motion in a knee joint of a patient for planning placement of a tibial implant on a tibia, the method comprising:
receiving a signal from a robotic arm indicating an establishment of communication with the robotic arm, the robotic arm being operatively connected to at least one muscle, wherein the at least one muscle is responsive to flexion in the knee joint such that rotation of the tibia relative to a femur of the patient using the robotic arm causes the at least one muscle to be stimulated;
collecting data at a plurality of positions of the tibia relative to a femur of the patient based on activation of the robotic arm, the plurality of positions collectively representative of at least part of a range of motion of the knee and including at least part of a distance between extension of the knee joint and flexion of the knee joint, the data at each position including:
a medial contact location defined by a maximum convergence of a low point on a medial condyle of the femur and a first surface location on a medial side of a tibial plateau of the tibial implant; and
a lateral contact location defined by a maximum convergence of a low point on a lateral condyle of the femur and a second surface location on a lateral side of the tibial plateau of the tibial implant;
analyzing the medial and lateral contact locations from the plurality of positions to determine a kinematic pattern of the knee joint; and
determining whether, at one or more positions, there is impingement between either the tibia and soft or hard tissue or the femur and soft or hard tissue,
wherein when there is impingement, modifying a planned tibial implant position to at least reduce an extent of impingement.
